(19) United States
(12) Patent Application Publication
(10) Pub. No.: US 2002/0010317 A1
(43) Pub. Date: Jan. 24, 2002

(54) Method for generating immunogens that elicit neutralizing antibodies against fusion-active regions of HIV envelope proteins

(76) Inventors: Carl T. Wild, Gaithersburg, MD (US); Graham P. Allaway, Darnestown, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/809,060

(22) Filed: Mar. 16, 2001

Related U.S. Application Data
(60) Provisional application No. 60/189,981, filed on Mar. 17, 2000.

(51) Int. Cl. A61K 39/21; C07K 14/16

(52) U.S. Cl. 530/350; 424/188.1; 435/235.1

(57) ABSTRACT

The current invention relates to methods of generating immunogens that elicit broadly neutralizing antibodies which target regions of viral envelope proteins such as the gp 120/gp41 complex of HIV-1. More specifically, the current invention involves using stabilizing peptides modeling the α-helical regions of the ectodomain of the HIV-1 transmembrane protein to stabilize fusion-active intermediate structures which can be used as vaccine immunogens.

### BACKGROUND OF THE INVENTION

### FIELD OF THE INVENTION

The present invention is related to HIV therapy and prophylaxis. In particular, the invention relates to methods for generating immunogens that elicit neutralizing antibodies against fusion-active regions of HIV-1 envelope proteins. Such methods, and pharmaceutical compositions therefor, can be employed to inhibit HIV infection.

### The HIV Envelope Proteins and HIV Cellular Receptors

The HIV-1 envelope glycoprotein is a 160 kDa glycoprotein that is cleaved to form the transmembrane (TM) subunit, gp41, which is non-covalently attached to the surface (SU) subunit, gp120 (Allan J. S., et al., Science 228:1091-1094 (1985); Veronese F. D., et al., Science 229:1402-1405 (1985)). Recent efforts have led to a clearer understanding of the structural components of the HIV-1 envelope system. Such efforts include crystallographic analysis of significant portions of both gp120 and gp41 (Kwong, P. D., et al., Nature (London) 393:648-659 (1998); Chan, D. C., et al., Cell 89:263-273 (1997); Weissenhorn, W., et al., Nature 387:426-430 (1997)).

The surface subunit has been structurally characterized as part of a multi-component complex consisting of the SU protein (the gp120 core absent the variable loops) bound to a soluble form of the cellular receptor CD4 (N-terminal domains 1 and 2 containing amino acid residues 1-181) and an antigen binding fragment of a neutralizing antibody (amino acid residues 1-213 of the light chain and 1-229 of the heavy chain of the 17b monoclonal antibody) which blocks chemokine receptor binding (Kwong, P. D., et al., Nature (London) 393:648-659 (1998)). Several envelope features believed to exist only in the fusion-active form of gp120 were revealed by the crystallographic analysis including a conserved binding site for the chemokine receptor, a CD4-induced epitope and a cavity-laden CD4-gp120 interface. This supports earlier observations of CD4-induced changes in gp120 conformation.

The gp120/gp41 complex is present as a trimer on the virion surface where it mediates virus attachment and fusion. HIV-1 replication is initiated by the high affinity binding of gp120 to the cellular receptor CD4 and the expression of this receptor is a primary determinant of HIV-1 cellular tropism in vivo (Dalgleish A. G., et al., Nature 312:763-767 (1984); Lifson J. D., et al., Nature 323:725-728 (1986); Lifson J. D., et al., Science 232:1123-1127 (1986); McDougal J. S., et al., Science 231:382-385 (1986)). The gp120-binding site on CD4 has been localized to the CDR2 region of the N-terminal V1 domain of this four-domain protein (Arthos, J., et al., Cell 5:469-481 (1989)). The CD4-binding site on gp120 maps to discontinuous regions of gp120 including the C2, C3 and C4 domains (Olshevsky, U., et al., Virol 64:5701-5707 (1990); Kwong, P. D., et al., Nature (London) 393:648-659 (1998)). Following attachment to CD4, the virus must interact with a “second” receptor such as a chemokine receptor in order to initiate the fusion process. Recently, researchers have identified the critical role of members of the chemokine receptor family in HIV entry (McDougal J. S., et al., Science 231:382-385 (1986); Feng Y., et al., Science 272:872-877 (1996); Alkhatib G., et al., Science 272:1955-1958 (1996); Doranz B. J., et al., Cell 85:1149-1158 (1996); Deng H., et al., Nature 381:661-666 (1996); Dragic T., et al., Nature 381:667-673 (1996); Choe H., et al., Cell 85:1135-1148 (1996); Dimitrov D. S., Nat. Med. 2:640-641 (1996); Broder, C. C. and Dimitrov, D. S., Pathobiology 64:171-179 (1996)). CCR5 is the chemokine receptor used by macrophage-tropic and many T-cell tropic primary HIV-I isolates. Most T-cell line-adapted strains use CXCR4, while many T-cell tropic isolates are dual tropic, capable of using both CCR5 and CXCR4.

Binding of gp120 to CD4 and a chemokine receptor initiates a series of conformational changes within the HIV envelope system (Eiden, L. E. and Lifson, J. D., Immunol. Today 13:201-206 (1992); Sattentau, Q. J. and Moore J. P., J. Exp. Med. 174:407-415 (1991); Allan J. S., et al., AIDS Res Hum Retroviruses 8:2011-2020 (1992); Clapham, P. R., et al., J. Virol. 66:3531-3537 (1992)). These changes occur in both the surface and transmembrane subunits and result in the formation of envelope structures which are necessary for virus entry. The functions of gp41 and gp120 appear to involve positioning the virus and cell membranes in close proximity thereby facilitating membrane fusion (Bosch M. L., et al., Science 244:694-697 (1989); Slepushkin V. A., et al., AIDS Res Hum Retroviruses 8:9-18 (1992); Freed E. O., et al., Proc. Natl. Acad. Sci. USA 87:4650-4654 (1990)).

### gp41

A good deal of structural information is available with respect to the HIV-1 transmembrane glycoprotein (gp41). This protein contains a number of well-characterized functional regions. See FIG. 3. For example, the N-terminal region consists of a glycine-rich sequence referred to as the fusion peptide which is believed to function by insertion into and disruption of the target cell membrane (Bosch, M. L., et al., Science 244:694-697 (1989); Slepushkin, V. A., et al., AIDS Res. Hum. Retrovirus 8:9-18 (1992); Freed, E. O., et al., Proc. Natl. Acad. Sci. USA 87:4650-4654 (1990); Moore, J. P., et al., “The HIV-cell Fusion Reaction,” in Viral Fusion Mechanism, Bentz, J., ed., CRC Press, Inc., Boca Raton, Fla. (1992)). Another region, characterized by the presence of disulfide linked cysteine residues, has been shown to be immunodominant and is suggested as a contact site for the surface (gp120) and transmembrane glycoproteins (Gnann, J. W., Jr., et al., J. Virol. 61:2639-2641 (1987); Norrby, E., et al, Nature 329:248-250 (1987); Xu, J. Y., et al., J. Virol. 65:4832-4838 (1991)). Other regions in the gp41 ectodomain have been associated with escape from neutralization (Klasse, P. J., et al., Virology 196:332-337 (1993); Thali, M., etal, J. Virol. 68:674-680 (1994); Stem, T. L., et al., J. Virol. 69:1860-1867 (1995)), immunosuppression (Cianciolo, G. J., et al., Immunol. Lett. 19:7-13 (1988); Ruegg, C. L., et al., J. Virol. 63:3257-3260 (1989)), and target cell binding (Qureshi, N. M., et al., AIDS 4:553-558 (1990); Ebenbichler, C. F., et al., AIDS 7:489-495 (1993); Henderson, L. A. and Qureshi, M. N., J. Biol. Chem. 268:15291-15297 (1993)).

Recent work has increased knowledge of the structural components of the HIV-1 transmembrane glycoprotein, however, the immunogenic nature of gp41 remains poorly understood. It is known that one of two immunodominant regions present in the HIV-1 envelope complex is located in gp41 (Xu, J. Y., et al., J. Virol. 65:4832-4838 (1991)). This region (TM residues 597-613) is associated with a strong, albeit non-neutralizing, humoral response in a large number of HIV+ individuals.

Two regions of the ectodomain of gp41 have been shown to be critical to virus entry. Primary sequence analysis predicted that these regions (termed the N-helix (residues 558-595 of the HIV-1_{LAI} sequence) and C-helix (residues 643-678 of the HIV-1^{LAI} sequence)) model α-helical secondary structure. Experimental efforts stemming from previous structural studies of synthetic peptide mimics established that the sequence analysis predictions were generally correct (Wild, C., et al., Proc. Natl. Acad. Sci. USA 89:10537-10541 (1992); Wild, C. T., et al., Proc. Natl. Acad. Sci. USA 91:9770-9774 (1994); Gallaher, W. R., et al., AIDS Res. Hum. Retroviruses 5:431-440 (1989); Delwart, E. L., et al., AIDS Res. Hum. Retroviruses 6:703-704 (1990)). Subsequent structural analysis determined that these regions of the transmembrane protein interact in a specific fashion to form a higher order structure characterized as a trimeric six-helix bundle (Chan, D. C., et al., Cell 89:263-273 (1997); Weissenhorn, W., etal., Nature 387:426-430 (1997)). This trimeric structure consists of an interior parallel coiled-coil trimer (region one) which associates with three identical α-helices (region two) which pack in an oblique, antiparallel manner into the hydrophobic grooves on the surface of the coiled-coil trimer. This hydrophobic self-assembly domain is believed to constitute the core structure of gp41. See FIGS. 4A and 4B.

While it has been demonstrated that the N- and C-helical regions of the transmembrane protein are critical to HIV-1 entry, their specific role in this process is unclear. It has been proposed that the association of these two regions to form the six-helix bundle core structure occurs during the transition from a nonfusogenic to a fusion-active form of gp41, and that the formation of this core structure facilitates membrane fusion by bringing the viral and target cell surfaces into close proximity (Chan, D. C. and Kim, P. S., Cell 93:681-684 (1998); FIG. 1). If correct, the formation of one or more structural intermediates necessary for viral fusion and entry, such as the six-helix bundle, is a key step in virus entry and factors which interfere with its formation could disrupt the entry event.

The effect of mutations in the N- and C-helical domains of gp41 provides additional clues as to the function of these regions in viral replication. Reports on the influence of structure-disrupting mutations in the N-helix on virus infectivity indicate that the structural components of gp41 are critical to viral entry (Wild, C., Proc. Natl. Acad. Sci. USA 91:12676-12680 (1994); Chen, S. S. -L, et al., J. Virol. 67:3615-3619 (1993); Chen, S. S. -L., J. Virol. 68:002-2010 (1994)). Further, sequence changes which decrease the structural stability of the N-helix coiled coil, result in an impaired fusion phenotype (Wild, C., Proc. Natl. Acad. Sci. USA 91:12676-12680 (1994)). Recently, Chen et al. demonstrated that coexpression of a mutant envelope defective for the N-helix structure with the wild-type envelope resulted in trans-dominant negative inhibition of virus replication (Chen, S. S. -L., et al., J. Virol. 72:4765-4774 (1998)).

Mutations in the N-helix of gp41 have also been shown to affect neutralization sensitivity. In such cases, neutralization is mediated by antibodies targeting the gp120 component of the envelope glycoprotein. An early report characterized a neutralization resistant escape mutant and identified a single amino acid substitution responsible for this change in phenotype (Klasse, P. J., et al., Virology 196:332-337 (1993)). Subsequent work identified a compensatory mutation which resulted in a return to the original phenotype (Stem, T. L., et al., J. Virol. 69:1860-1867 (1995)). The mutation resulting in escape was in the N-helix while the compensatory change was in the C-helix which is consistent with the proposed cooperative interaction of these regions of gp41.

Recently, Park and Quinnan identified several changes in the N-helical domain which resulted in an alteration in both infectivity and neutralization sensitivity (Park, E. J., et al., J. Virol. 72:7099-7107 (1998); Park, E. J. and Quinnan, G. V., Jr., J. Virol. 73:5707-5713 (1999)). In both instances, it was speculated that changes in gp41 affect gp120-mediated neutralization by altering the structure of the surface protein. While it is unclear how mutations in one subdomain might affect structure in the other, it has been proposed that contacts between the C-terminus of gp120 and the N-terminus of gp41 could serve to transfer the effect.

Mutations in the C-helix of gp41 have also been analyzed for their affect on viral entry. Salzwedel et al. showed that deletions, substitutions and insertions centered around a tryptophan-rich stretch of 17 amino acid residues overlapping the carboxy-terminus of the C-helix affected the ability of gp41 to mediate fusion (Salzwedel, K., et al., J. Virol. 73:2469-2480 (1999)). From their results, they concluded that this tryptophan-rich motif plays a critical role in a post-CD4-binding step necessary for membrane fusion.

### Vaccine Development

Developing a vaccine against HIV is a major worldwide goal for disease prevention. However, despite intense efforts, an effective vaccine candidate has proven to be an elusive target, in part because of the considerable genetic variation within and between HIV-1 isolates. An additional hurdle is the incomplete understanding of protective immunity. Theory and experimental data support the idea that inducing a broadly neutralizing antibody response would have value in preventing or limiting HIV infection. For example, protection of macaques from SIV infection following immunization with live attenuated SIV appears to be, in part, mediated by a humoral antibody response (Wyand, M. S., et al., J. Vitol. 70:3724-3733 (1996)). It has also been demonstrated that chimpanzees can be protected from infection by a laboratory-adapted strain of HIV-I following passive administration of a V3-directed monoclonal antibody (Emini, E. A., et al., Nature 355:728-730 (1992)). Thus, a focus of the invention is to generate and characterize a humoral immune response targeting fusion-active forms of the HIV envelope.

The HIV-1 envelope glycoproteins (gp160, gp120 and gp⁴1) have been shown to be the major antigens for anti-HIV antibodies present in AIDS patients (Barin, et al., Science 228:1094-1096 (1985)). Thus far, these proteins seem to be the most promising candidates to act as immunogens for anti-HIV vaccine development. To this end, several groups have begun to use various portions of gp160, gp120 and/or gp41 as immunogenic targets for the host immune system. Although these attempts have met with minimal success, researchers have observed that the humoral response generated against native forms of the viral envelope (primarily oligomeric forms of the gp120/gp41 complex) is more broadly neutralizing than antibody raised against denatured and/or monomeric viral envelope (VanCott, T. C., et al., J. Vitol. 71:4319-4330 (1997)). This supports the concept that viral structure is critical for both understanding the immunogenicity of envelope proteins and designing envelope-based immunogens which induce a broad neutralizing response against HIV.

The epitope for the broadly neutralizing monoclonal antibody 2F5 is located adjacent to the membrane-spanning domain in a transmembrane region which is rich in hydrophobic and uncharged residues (transmembrane protein residues 662-667) (Muster, T., et al., J. Virol. 67:6642-6647 (1993); Muster, T., et al., J. Vitol. 68:4031-4034 (1994)). It is interesting to note that 2F5 maps to a determinant of the transmembrane protein that overlaps one of the two regions of gp41 which interact to form the hydrophobic core of the protein. This observation has lead to speculation that 2F5 might actually neutralize virus by interacting with and disrupting the function of an entry-relevant gp41 structure. An extensive study which mapped the antigenic structure of gp41 supports this idea. This work characterized several conformation dependent gp41 monoclonal antibodies (MAbs) which mapped to the same region of the transmembrane protein as 2F5 (Earl, P. L., et al., J. Vitol. 71:2647-2684 (1997)). Although the binding sites for these non-neutralizing MAbs overlapped the 2F5 determinant, in competition experiments none of the non-neutralizing antibodies were blocked from binding to the native protein by the 2F5 MAb. This indicates that, while the two dimensional regions to which these antibodies map are similar, the three dimensional epitopes to which they bind are quite different.

The observation that only one neutralizing MAb, 2F5, maps to the ectodomain of gp41 and that antibodies to the 2F5 epitope are poorly represented in sera from HIV-infected individuals suggest that, for the most part, gp41 neutralizing epitopes are cryptic. The cryptic nature of these neutralizing epitopes is most likely related to the functional role of the transmembrane protein in HIV-1 replication which involves mediating virus entry.

### Related Art

U.S. Pat. No. 5,464,933 and PCT Publication No. 94/28920, Bolognesi et al., describe peptides which exhibit anti-retroviral activity. Specifically disclosed is the peptide DP-178 derived from the HIV-1_{LAI} gp41 protein, as well as fragments, analogs and homologs of DP-178. The peptides are used as inhibitors of human and non-human retroviral transmission to uninfected cells.

U.S. Pat. No. 5,656,480 and PCT Publication No. WO 94/02505, Wild et al., describe protein fragments derived from the HIV transmembrane glycoprotein (gp41), including the peptide DP-107, which have antiviral activity. Also disclosed are methods for inhibiting enveloped viral infection, and methods for modulating biochemical processes involving coiled coil peptide interactions.

PCT Publication No. WO 96/40191, Johnson et al., describes compositions used to treat or prevent viral infections, including HIV infections. The compositions contain DP-178 or DP-107 in combination with another anti-viral therapeutic agent.

PCT Publication No. WO 96/19495, Bolognesi et al., is directed to anti-retroviral peptides including DP-178- and DP-107-related peptides recognized by specific computer sequence search motifs. The peptides are used to inhibit viral transmission to a cell.

### SUMMARY OF THE INVENTION

The present invention relates to a vaccine that provides a protective response in an animal comprising one or more immunogens of the present invention together with a pharmaceutically acceptable diluent, carrier or excipient, wherein the vaccine may be administered in an amount effective to elicit an immune response in an animal to a virus. In one embodiment, the animal is a mammal such as a human. In another embodiment, the virus is HIV. In another embodiment, the virus is HIV-1.

The present invention also relates to methods for forming immunogens of the invention.

The present invention also relates to immunogenic compositions comprising at least one immunogen of the invention and a pharmaceutically acceptable diluent, carrier or excipient.

In alternative embodiments, the invention relates to an immunogenic composition comprising at least one viral envelope protein or fragment thereof exterior to the viral membrane, and at least one gp41 α-helical peptide (N-helix or C-helix) (stabilizing peptide), and, optionally, at least one viral cell surface receptor, wherein the α-helical peptide is capable of associating with the envelope protein or fragment thereof to form a stable structure.

The invention further relates to an immunogenic composition produced by a process, which comprises incubating at least one non-infectious viral particle with one or more stabilizing peptides to obtain a mixture and adding a soluble form of one or more viral cell surface receptors to the mixture in an amount sufficient to activate the envelope for viral entry, whereby an immunogenic composition is created. Preferably, the stabilizing peptide is present in an amount effective to disrupt the formation by viral envelope protein in the presence of soluble or membrane-bound CD4 of one or more structural intermediates necessary for viral fusion and entry, for example, the six-helix bundle.

The invention further relates to a method of preparing an immunogenic composition, which comprises incubating at least one non-infectious viral particle having at least one surface envelope protein or fragment thereof exterior to the viral membrane with at least one stabilizing peptide to obtain a protein/peptide first mixture, adding a soluble form of at least one cell surface receptor or fragment thereof to the protein/peptide first mixture in an amount sufficient to activate the protein or fragment thereof for viral entry to create a second mixture, and isolating the resultant fusion-active protein/peptide complex from the second mixture. Preferably, the stabilizing peptide is present in an amount effective to disrupt the formation of one or more structural intermediates necessary for viral fusion and entry by viral envelope protein in the presence of soluble or membrane-bound CD4.

The invention further relates to a method of preparing an immunogenic composition, which comprises incubating cells expressing at least one HIV envelope protein or fragment thereof exterior to the viral membrane with at least one stabilizing peptide to obtain a protein/peptide first mixture, adding a soluble form of at least one cell surface receptor or fragment thereof to the protein/peptide first mixture in an amount sufficient to activate the at least one protein or fragment thereof for viral entry to create a second mixture, isolating the resultant fusion-active protein/peptide complex from the second mixture by treating the second mixture with a lysis buffer, and purifying the protein/peptide complex. Preferably, the stabilizing peptide is present in an amount effective to disrupt the formation of one or more structural intermediates necessary for viral fusion and entry by viral envelope protein in the presence of soluble or membrane-bound CD4.

The invention further relates to a method of preparing vaccine immunogens, which comprises introducing structure disrupting mutations into specific positions in the structured regions of gp41 or fragment thereof, wherein the mutations result in constructs which expose isolated forms of the N- and/or C-helical regions which, in the wild-type envelope protein, are transient in nature and exist only during the period immediately following receptor binding, but prior to six-helix bundle formation. The mutations result in the production of a fusion-active vaccine immunogen.

In one embodiment, the mutations comprise substitutions of the invariant residues within the 4-3 heptad repeats found in each helical region with residues incompatible with the formation of α-helical secondary structure.

The invention further relates to a product formed by any of the above methods.

### BRIEF DESCRIPTION OF THE FIGURES

FIG. 1 illustrates the role of gp41 in mediating virus entry. In the native state, the HIV-1 envelope complex exists in a nonfusogenic form. Following CD4 (and in some cases chemokine) binding, the pre-hairpin intermediate forms. At this point, the transmembrane protein, gp41, is in an extended conformation and the N- and C-helical domains have yet to associate. In the absence of a stabilizing peptide, this intermediate proceeds to form the six-helix bundle (hairpin intermediate). It is proposed that the formation of the bundle serves to facilitate virus-target cell fusion by drawing the viral and cellular membranes close together. In the presence of a stabilizing peptide, the pre-hairpin intermediate is stabilized by the interaction of the peptide with its complementary region of gp41. The stabilized pre-hairpin intermediate is one form of the fusion-active immunogens described in this application.

FIGS. 2A-2C illustrate the use of an epitope-tagged version of DP-178 (DP-178HA) to capture and stabilize a fusion-active form of gp41. FIG. 2A shows co-immunoprecipitation of gp41 by DP-178HA following HXB2 envelope activation by binding to soluble and cell expressed CD4 (+/− indicates presence or absence of CD4). FIG. 2B shows the blocking of co-immunoprecipitation of DP-178HA binding by an anti-CD4 binding antibody (Q4120, Sigma). FIG. 2C shows the effect of receptor activation (both CD4 and chemokine) on HIV-1 primary, CCR5-dependent isolate envelopes. In each panel, * indicates bands due to IgG heavy chain and ** indicates bands due to shorter fragments of gp41 probably resulting from proteolysis.

FIG. 3 is a schematic representation of the structural and antigenic regions of HIV-1 gp41.

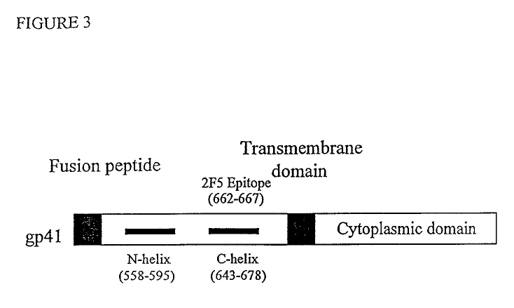

FIGS. 4A and 4B are schematic representations of the interaction of the N- and C-helical domains of gp41 to form the six-helix bundle structure. Both top and side views are shown. The interior of the bundle represents the N-helical coiled-coil. The exterior components represent the C-helical domain.

FIG. 5 is a schematic representation of the proposed gp41 intermediate structures formed during virus entry. Fusion intermediate I forms immediately following receptor binding and shows the ectodomain in an extended form. Fusion intermediate II shows gp41 following core structure formation. The stabilizing peptides are believed to inhibit by interacting with the complementary regions of gp41 in a dominant-negative fashion.

FIG. 6 depicts the effect of point mutations in the N- and C- domains of gp41 on the intermediate structure. The fusion intermediate containing structure-disrupting mutations in the N-helix presents the C-helical region in its isolated fusion-active form. The fusion intermediate containing structure-disrupting mutations in the C-helix presents the N-helical region in its isolated fusion-active form.

FIGS. 7A and 7B are graphs illustrating percent neutralization for gp233 and gp234 sera in different experimental formats. FIG. 7A shows the titration of bleed 2 for each animal against HIV-1_{MN} in a cell killing assay which uses cell viability as a measure of virus neutralization. MT-2 cells are added to a mixture of virus (sufficient to result in >80% cell death at 5 days post infection) and sera which had been allowed to incubate for about 1 hour. After 5 days in culture, cell viability was measured by vital dye metabolism. FIG. 7B shows the percent neutralization for each bleed at a 1:10 dilution against HIV-1_{MN} in an assay format employing CEM targets and p24 endpoint. In this assay, sera were incubated with 200 TCID₅₀ of virus for about 1 hour prior to the addition of the cells. On days 1, 3 and 5, media were changed. On day 7, culture supernatants were collected and analyzed for virus replication by p24 antigen levels. In each assay format, percent neutralization was determined by comparing experimental wells with cell and cell/virus controls.

### DETAILED DESCRIPTION OF THE PREFERRED EMBODIMENTS

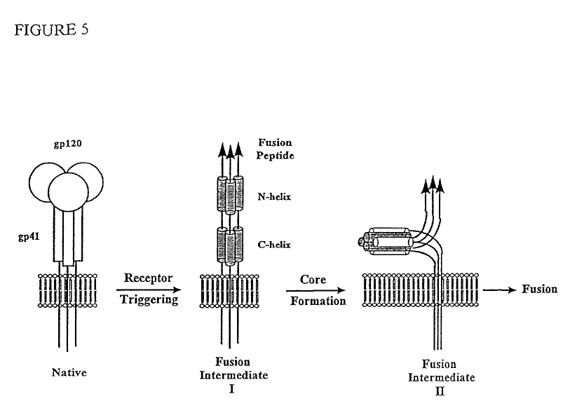

As described above, the initial, and best understood, step in the HIV entry process involves the binding of the gp120 subunit to CD4. Prior to the binding of the virus to the target cell receptor, i.e., gp120-CD4 binding, the viral envelope complex (gp41/gp120) exists in a nonfusogenic form. The viral envelope complex is referred to as fusion-active following attachment of the virus to the host cell whereby the entry structures in envelope complex are formed and/or exposed. The binding event triggers receptor-mediated conformational changes involving both gp120 and gp41. Specifically, binding results in the formation of a series of structural intermediates termed “early fusion-active” intermediates which mediate the formation of the well-characterized six-helix bundle (Furuta, R. A., et al., Nature Structural Biol. 5:276-279 (1998)). Since the structural intermediates form and function only during virus entry and drive the conformational changes required for virus entry, they are believed to be critical to virus entry (FIG. 5). For some HIV strains, binding to CD4 is sufficient to trigger the formation of one or more structural intermediates necessary for viral fusion and entry while for other HIV strains, binding to a secondary receptor (usually the CCR5 or the CXCR4 chemokine receptor) is required. The fusion-active structural intermediates constitute a novel set of neutralizing epitopes within HIV gp120/gp41.

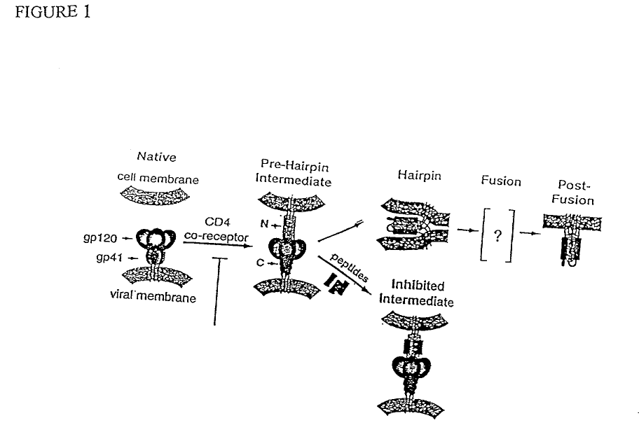

It has been shown that peptides which model the a-helical structural components from the gp41 N- and C-helical regions exhibit potent antiviral activity (Wild, C., et al., Proc. Natl. Acad. Sci. USA 89:10537-10541 (1992); Wild, C. T., et al., Proc. Natl. Acad. Sci. USA 91:9770-9774 (1994)). Termed DP-107 and DP-178, these compounds, which are disclosed in U.S. Pat. Nos. 5,464,933 and 5,656,410, have been shown to be virus specific inhibitors of HIV-1 replication that function at the level of virus entry (FIG. 1) (Wild, C., et al., Proc. Natl. Acad. Sci. USA 89:10537-10541 (1992); Wild, C. T., et al., Proc. Natl. Acad. Sci. USA 91:9770-9774 (1994)). The most effective of these, DP-178, inhibits envelope mediated cell-cell fusion at concentrations as low as about 1 ng/ml. Although less potent, DP-107 also inhibits cell-cell fusion at sub-μg/ml levels. These compounds are equally effective against a wide variety of laboratory adapted and primary virus isolates representing a range of subtypes.

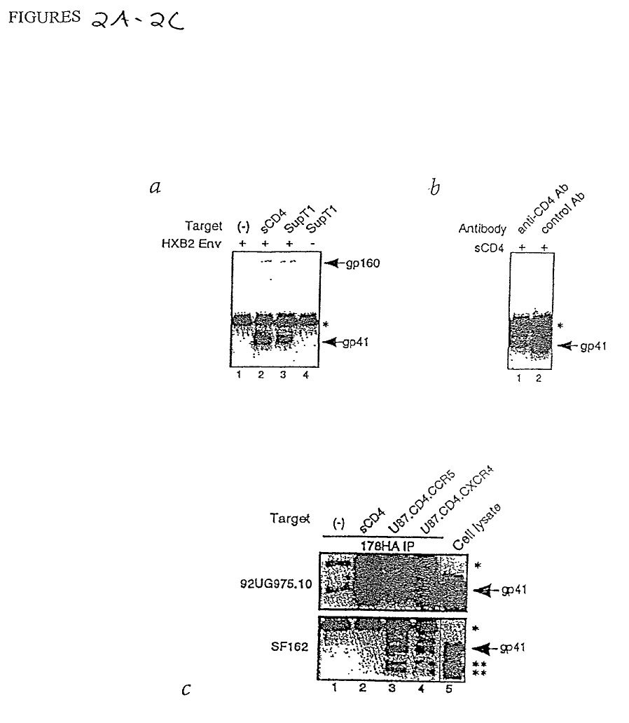

The observation that DP-107 and DP-178 inhibit virus replication at the level of viral entry has led to speculation that the peptides inhibit in a dominant-negative manner. Immediately following CD4 binding, the N- and C-helical components of gp41 have yet to associate. It is during this time that the DP-178 or DP-107 peptide is able to interact with the complementary native envelope determinant and disrupt core structure formation. In the absence of DP-178, DP-107 or a functionally similar inhibitor, the N- and C-helical domains associate to form the six-helix complex (FIG. 1). This is supported by structural studies which show that peptides modeling these regions of gp41 combine in vitro to form the six-helix bundle and the recent finding that the DP-178 peptide binds to a fusion-active form of gp41 (Furuta, R. A., et al., Nature Structural Biol. 5:276-279 (1998)). More specifically, it has been demonstrated that the peptide DP-178 inhibits virus entry by “freezing” gp41 in an early fusion-active form (FIG. 2).

The current invention involves using the stabilized fusion-active envelope structures as vaccines. More specifically, the current invention relates to methods of generating immunogens that elicit broadly neutralizing antibodies which target regions of HIV envelope proteins, specifically, proteins such as the gp120/gp41 complex. In one embodiment, the current invention involves using stabilizing peptides modeling the α-helical regions of the ectodomain of the HIV transmembrane protein to stabilize fusion-active intermediate structures.

### Fusion-Active Vaccine Immunogens

The invention is directed to stabilizing peptides modeling the N- and C-helical domains that are capable of interacting in a dominant-negative fashion with native viral protein. This peptide/protein interaction serves to “freeze out” or trap stable gp41 entry intermediates. Combinations of viral proteins and stabilizing peptides can be used to generate stabilized forms of fusion-active gp41 for use as vaccine immunogens. The invention is also directed to the introduction of mutations into specific positions in the viral transmembrane protein. These envelope mutants form stable fusion-active structures which can be employed as vaccine immunogens.

Specifically, the present invention relates to an immunogenic composition comprising at least one viral envelope protein or fragment thereof exterior to the viral membrane and an amount of at least one stabilizing peptide effective to disrupt the formation of one or more structural intermediates necessary for viral fusion and entry and, optionally, at least one viral cell surface receptor or fragment thereof, wherein the stabilizing peptide is capable of associating with the envelope protein or fragment thereof to form a stabilized, fusion-active structure. The stabilized, fusion-active structure is also referred to as a stabilized pre-hairpin intermediate. Thus, at least two types of vaccine immunogens are generated including an immunogen containing the complete mixture (protein/receptor/peptide), and an immunogen containing the protein/peptide complex which will be released from the mixture by lysis, for example, and recovered by affinity chromatography, for example, as described below.

In one embodiment, the at least one viral envelope protein or fragment thereof is a protein or fragment thereof exterior to the viral membrane. In another embodiment, the protein or fragment thereof is the HIV-1 gp41/gp120 complex or fragment thereof.

In another embodiment, the at least one viral cell surface receptor or fragment thereof is an HIV-1 cell surface receptor such as CD4 or fragment thereof, optionally attached to a fusion protein. The fragments include at least the V1 domain of CD4 with the presence of the V1 and V2 domains being preferred. Cell surface receptors can be obtained from a cell line that (a) expresses CD4 or a fragment thereof as described above, (b) expresses a membrane preparation that expresses or contains CD4 or fragment thereof as described above, or (c) expresses an appropriate chemokine receptor such as CCR5, CXCR4 or mixtures thereof; or (d) expresses combinations of (a), (b) and/or (c).

Useful stabilizing peptides are selected from the group consisting of: a peptide comprising SEQ ID NO:1, a peptide comprising a fragment of SEQ ID NO:1, a peptide comprising SEQ ID NO:2, a peptide comprising a fragment of SEQ ID NO:2, a peptide comprising SEQ ID NO:3, a peptide comprising a fragment of SEQ ID NO:3, a peptide comprising SEQ ID NO:4, a peptide comprising a fragment of SEQ ID NO:4, a peptide comprising SEQ ID NO:5, a peptide comprising a fragment of SEQ ID NO:5, a peptide comprising SEQ ID NO:6, a peptide comprising a fragment of SEQ ID NO:6, a peptide comprising SEQ ID NO:7, a peptide comprising a fragment of SEQ ID NO:7, a peptide comprising SEQ ID NO:9, a peptide comprising a fragment of SEQ ID NO:9, a peptide comprising any combination of SEQ ID NOS:1-7 and 9, a peptide comprising any combination of fragments of SEQ ID NOS:1-7 and 9, a peptide functionally equivalent to any one of SEQ ID NOS:1-7 and 9, a homolog of any of SEQ ID NOS:1-7 and 9, an analog of any of SEQ ID NOS:1-7 and 9 and mixtures thereof. Additional useful peptides are further described herein.

The invention further relates to an immunogenic composition produced by a process, which comprises incubating at least one non-infectious viral particle with a concentration of one or more stabilizing peptides effective to disrupt the formation of one or more structural intermediates necessary for viral fusion and entry to obtain a mixture and adding a soluble form of one or more viral cell surface receptors or fragments thereof to the mixture in an amount sufficient to activate viral entry, whereby an immunogenic composition is created.

The invention further relates to a method of preparing an immunogenic composition, which comprises incubating at least one non-infectious viral particle having at least one surface envelope protein or fragment thereof exterior to the viral membrane with an effective amount of at least one stabilizing peptide to obtain a protein/peptide first mixture, adding a soluble form of at least one cell surface receptor or fragment thereof to the protein/peptide first mixture, and isolating the resulting fusion-active peptide complex from the second mixture. The peptide complex can be isolated from the second mixture by methods known in the art, such as treating the mixture with a detergent. The peptide complex can optionally be purified using methods known in the art, such as ion exchange chromatography, affinity chromatography, ultracentrifugation or gel filtration. The resulting complex can function effectively as a vaccine immunogen.

In one embodiment, the at least one surface envelope protein or fragment thereof is the HIV-1 gp41/gp120 complex or fragment thereof.

In another embodiment, the at least one cell surface receptor or fragment thereof is an HIV-1 cell surface receptor such as CD4 or fragment thereof, optionally attached to a fusion protein. The fragments include at least the V1 domain of CD4 with the presence of the V1 and V2 domains being preferred. The at least one cell surface receptor can be obtained from a cell line that (a) expresses CD4 or a fragment thereof as described above, (b) expresses a membrane preparation that expresses or contains CD4 or fragment thereof as described above, or (c) expresses an appropriate chemokine receptor such as CCR5, CXCR4 or mixtures thereof; or (d) expresses combinations of (a), (b) and/or (c).

Useful stabilizing peptides are selected from the group consisting of: a peptide comprising SEQ ID NO:1, a peptide comprising a fragment of SEQ ID NO:1, a peptide comprising SEQ ID NO:2, a peptide comprising a fragment of SEQ ID NO:2, a peptide comprising SEQ ID NO:3, a peptide comprising a fragment of SEQ ID NO:3, a peptide comprising SEQ ID NO:4, a peptide comprising a fragment of SEQ ID NO:4, a peptide comprising SEQ ID NO:5, a peptide comprising a fragment of SEQ ID NO:5, a peptide comprising SEQ ID NO:6, a peptide comprising a fragment of SEQ ID NO:6, a peptide comprising SEQ ID NO:7, a peptide comprising a fragment of SEQ ID NO:7, a peptide comprising SEQ ID NO:9, a peptide comprising a fragment of SEQ ID NO:9, a peptide comprising any combination of SEQ ID NOS:1-7 and 9, a peptide comprising any combination of fragments of SEQ ID NOS:1-7 and 9, a peptide functionally equivalent to any one of SEQ ID NOS:1-7 and 9, a homolog of any of SEQ ID NOS:1-7 and 9, an analog of any of SEQ ID NOS:1-7 and 9, the influenza hemagglutinin epitope, an epitope-tagged peptide and mixtures thereof. Additional useful peptides are further described herein.

The invention further relates to a method of preparing an immunogenic composition, which comprises incubating cells expressing at least one HIV envelope protein or fragment thereof exterior to the viral membrane with an effective amount of at least one stabilizing peptide to obtain a protein/peptide first mixture, adding a soluble form of at least one cell surface receptor or fragment thereof to the protein/peptide first mixture in an amount sufficient to create a second mixture, isolating the resulting fusion-active peptide complex from the second mixture by treating the second mixture with a lysis buffer, and purifying the peptide/envelope complex. The peptide/envelope complex can be purified using methods known in the art, such as affinity chromatography, ion exchange chromatography, ultracentrifugation or gel filtration. The resulting complex can function effectively as a vaccine immunogen.

In another embodiment, the cells expressing the at least one HIV envelope protein or fragment thereof are cells infected with a recombinant vaccinia virus expressing the HIV-1 envelope protein or fragment thereof.

In another embodiment, the cells expressing the at least one HIV envelope protein or fragment thereof are cells transformed with a vector expressing the HIV-1 envelope protein or fragment thereof.

Useful stabilizing peptides are the selected from the group consisting of: a peptide comprising SEQ ID NO:1, a peptide comprising a fragment of SEQ ID NO:1, a peptide comprising SEQ ID NO:2, a peptide comprising a fragment of SEQ ID NO:2, a peptide comprising SEQ ID NO:3, a peptide comprising a fragment of SEQ ID NO:3, a peptide comprising SEQ ID NO:4, a peptide comprising a fragment of SEQ ID NO:4, a peptide comprising SEQ ID NO:5, a peptide comprising a fragment of SEQ ID NO:5, a peptide comprising SEQ ID NO:6, a peptide comprising a fragment of SEQ ID NO:6, a peptide comprising SEQ ID NO:7, a peptide comprising a fragment of SEQ ID NO:7, a peptide comprising SEQ ID NO:9, a peptide comprising a fragment of SEQ ID NO:9, a peptide comprising any combination of SEQ ID NOS:1-7 and 9, a peptide comprising any combination of fragments of SEQ ID NOS:1-7 and 9, a peptide functionally equivalent to any one of SEQ ID NOS:1-7 and 9, a homolog of any of SEQ ID NOS:1-7 and 9, an analog of any of SEQ ID NOS:1-7 and 9, influenza hemagglutinin epitope, an epitope-tagged peptide and mixtures thereof.

In another embodiment, the at least one cell surface receptor or fragment thereof is obtained from a cell line that (a) expresses CD4 or fragment thereof as described below, (b) expresses a membrane preparation that expresses or contains CD4 or fragment thereof as described below, or (c) expresses an appropriate chemokine receptor such as CCR5, CXCR4 or mixtures thereof. Cell lines that express combinations of (a) and (c) or (b) and (c) are also contemplated. Fragments of CD4, optionally attached to a fusion protein, are included. Fragments include at least the V1 domain of CD4 with the presence of the V1 and V2 domains being preferred.

In another embodiment, the at least one HIV envelope protein or fragment thereof is a recombinant form of the HIV-1 gp41 ectodomain.

In another embodiment, the receptor/peptide/envelope complex is formed in the presence of a denaturant.

The invention further relates to a product formed by any of the above methods.

### Preparation of Fusion-Active Vaccine Immunogens

In general, the fusion-active vaccine immunogens can be formulated in ways that are minimally disruptive to structural components while optimizing immunogenicity. The preparation of the immunogens involves incubating at least one non-infectious viral particle or pseudovirion bearing at least one envelope protein or fragment thereof from at least one laboratory-adapted or primary viral isolate with a concentration of at least one stabilizing peptide effective to disrupt the formation of one or more structural intermediates necessary for viral fusion and entry. Following incubation, a soluble form of at least one viral receptor or fragment thereof is added. The addition of the viral receptor or fragment thereof activates the envelope protein or fragment thereof for viral entry. Without wishing to be bound by theory, the at least one stabilizing peptide then binds and locks the envelope protein or fragment thereof in its fusion-active form. The resulting fusion active peptide complex forms the inventive vaccine immunogen. The fusion active peptide/envelope complex can be further treated to isolate the specific peptide/envelope complex from other components of the mixture by treating the mixture with a detergent to disrupt the lipid membrane in which the envelope protein is embedded, and then purifying the detergent-treated mixture using, e.g., ion exchange chromatography, gel filtration, affinity chromatography or ultracentrifugation.

More specifically, one method of preparing the vaccine immunogens of the invention involves incubating at least one a non-infectious HIV-1 particle (an example being 8E5/LAV virus (Folks, T. M., et al., J. Exp. Med. 164:280-290 (1986); Lightfoote, M. M., et al, J. Vitol. 60:771-775 (1986); Gendelman, H. E., etal., Virology 160:323-329 (1987))) or pseudovirion bearing the HIV envelope glycoprotein or fragment thereof from at least one laboratory-adapted or primary HIV-1 isolate (Haddrick, M., et al., J. Vitol. Methods 61:89-93 (1996); Yamshchikov, G. V., et al., Virology 21:50-58 (1995)) with a concentration of at least one stabilizing peptide effective to disrupt the formation of one or more structural intermediates necessary for viral fusion and entry such as P-17 (SEQ ID NO:6), P-18 (SEQ ID NO:1), a peptide comprising a combination of P-17 and P-18, a peptide comprising a combination of fragments of P-17 and P-18, a peptide comprising P-17 or a fragment thereof, a peptide comprising P-18 or a fragment thereof, or a peptide functionally similar to P-17 and/or P-18.

Preferably, in each of the embodiments of the present invention the stabilizing peptide and the envelope protein have a molar ratio of from about 0.1 moles to about 100 moles of stabilizing peptide per mole of envelope protein. Most preferably, the molar ratio is about 0.5 to about 10 moles of stabilizing peptide per mole of envelope protein.

The 8E5/LAV cell line produces an intact virion expressing functional envelope in a non-replicating system. Following incubation of the virion with a peptide, a soluble form or fragment thereof of the primary HIV-1 receptor, CD4, is added (sCD4). The addition of sCD4 activates the envelope protein or fragment thereof for viral entry by binding to and triggering gp120 which in turn will allow the stabilizing peptide to capture the newly exposed fusion-active form of gp41.

In an alternative embodiment, a recombinant form of the gp41 ectodomain (AA residues 527-670 HXB2 numbering) is incubated with the C- or N-helical stabilizing peptides under denaturing conditions followed by slow re-folding. The denaturant will disrupt native protein structure (the recombinant has been shown to model the native six-helix bundle) and allow the peptide to interact with the complementary gp41 determinants. Refolding will give rise to a peptide/gp41 complex which represents either entry domain in its early fusion-active form.

In an alternative embodiment, the at least one stabilizing peptide used to form the fusion-active structure can be synthesized to contain, for example, the influenza hemagglutinin epitope at the C-terminus. The peptide/envelope complex can then be purified using an affinity column generated with a monoclonal antibody specific for, for example, the influenza hemagglutinin epitope (Furuta, R. A., et al., Nature Structural Biol. 5:276-279 (1998)).

In another alternative embodiment, cells expressing the at least one viral envelope protein, e.g., cells infected with a recombinant vaccinia virus expressing the HIV-1 envelope protein or fragment thereof (Earl, P. L., etal., J. Vitol. 65:31-41 (1991); Rencher, S. D., et al., Vaccine 5:265-272 (1997); Katz, E. and Moss, B., AIDS Res. Hum. Retroviruses 13:1497-1500 (1997)), can be used. The addition of sCD4 then activates the envelope protein or fragment thereof for viral entry by binding to and triggering gp120 which in turn will allow the stabilizing peptide to capture the newly exposed fusion-active form of gp41. The envelope-expressing cells can be incubated with a concentration of the at least one stabilizing peptide effective to disrupt the formation of one or more structural intermediates necessary for viral fusion and entry. Following treatment with a lysis buffer, the envelope protein/peptide complex can be purified using the methods described above.

The envelope-expressing cells can be incubated for approximately one hour, for example, under physiologic conditions, with a concentration effective to disrupt the formation of one or more structural intermediates necessary for viral fusion and entry of P-17 (SEQ ID NO:6), P-18 (SEQ ID NO:1), a peptide comprising P-17 or a fragment thereof, a peptide comprising P-18 or a fragment thereof, a peptide comprising a combination of P-17 and P-18, a peptide comprising a combination of fragments of P-17 and P-18, a peptide functionally similar to P-17 and/or P-18 or an epitope-tagged peptide, and then treated with sCD4 and a lysis buffer such as 1% Triton X-100, 150 mM NaCl, 50 mM Tris-Cl, pH 7.4. The concentration of the epitope-tagged peptide would be approximately two-fold higher than the non-tagged version. A specific peptide may be P-18-GGG-YPYDVPDYAGPG, wherein the epitope tag is in bold.

Following treatment with the lysis buffer, the peptide/envelope protein complex can be purified using the methods described above. The epitope tag may be added to the C-terminus of the peptide during synthesis and may correspond to a determinant in the influenza virus hemagglutinin protein. A monoclonal antibody specific for this epitope is commercially available.

As another alternative embodiment, in the methods described above, CD4 and chemokine expressing cell lines can be substituted for sCD4. By this method, the at least one non-infectious virion or the envelope-expressing cell would be incubated under physiologic conditions for approximately one hour, for example, with the at least one stabilizing peptide or epitope-tagged peptide, and then incubated with a cell line expressing CD4 or fragment thereof, optionally attached to a fusion protein, or expressing a membrane preparation that expresses or contains CD4 or fragment thereof as described above. The fragments include at least the V1 domain of CD4 with the presence of the V1 and V2 domains being preferred. Alternatively, the cell line may express an appropriate chemokine receptor such as CCR5 or CXCR4, or may express a combination of CD4 and chemokine receptors or fragments thereof. Following treatment with a lysis buffer, the envelope protein/peptide complex can be purified as previously described.

As another alternative embodiment, a recombinant form of the HIV-1 gp41 ectodomain expressed in, e.g., bacterial or mammalian cells, could be incubated for approximately one hour, for example, at room temperature, for example, with a concentration effective to disrupt the formation of one or more structural intermediates necessary for viral fusion and entry of at least one stabilizing peptide under denaturing conditions such as, for example, 6 M GuHCl or 8 M urea. Optionally, the protein could be heated for about thirty minutes, for example, at about 70° C., for example. The denaturant may be removed by dialysis of the resulting peptide/gp41 complex against distilled water. Further dialysis steps may be conducted to allow for slow refolding of the protein. The resulting complex of the recombinant gp41 and at least one stabilizing peptide constitutes a vaccine immunogen.

The methods described above can be applied to other viruses where the envelope proteins form similar complexes that are critical to virus entry including, but not limited to, HIV-2, HTLV-I, HTLV-II, feline immunodeficiency virus (FIV), human parainfluenza virus III (HPV-III), respiratory syncytial virus (RSV), human influenza virus, measles virus, and combinations thereof.

### The Effect of Mutations on gp41 Entry Determinants

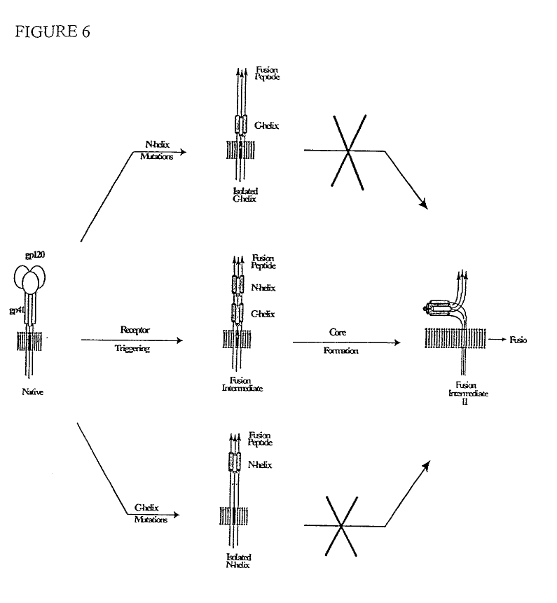

An alternative method for preparing vaccine immunogens presenting stable early fusion-active gp41 structures is site specific mutagenesis. This approach involves the introduction of mutations into specific positions in the structural regions of the viral transmembrane protein. These mutations will result in constructs which present isolated forms of the N- and/or C-helical regions which, in the wild-type envelope protein, are transient in nature and exist only during the period immediately following receptor binding, but prior to six-helix bundle formation (FIG. 6). This maybe accomplished by introducing structure disrupting mutations into the N- and C-helical regions of gp41 or a fragment thereof. Disrupting the structural components in either of these highly conserved elements of gp41 will result in a fusion-active immunogen which represents the remaining α-helical component in its isolated form.

The mutations involve substitutions of the invariant residues within the 4-3 heptad repeats found in each helical region with residues incompatible with the formation of α-helical secondary structure. In most cases, this approach efficiently abrogates structure without disrupting envelope expression (Wild, C., Proc. Natl. Acad. Sci. USA 91:12676-12680 (1994)). For example, a leucine or isoleucine may be replaced by a known helix breaker such as glycine. Initially, the effect of each proposed mutation on helical structure may be determined using synthetic peptides. The changes which result in significant disruption of peptide secondary structure may be incorporated into a eucaryotic expression vector and characterized for their effect on protein secondary structure using a surface immunoprecipitation assay employing antibodies specific for the six-helix bundle. The constructs which are deficient for core structure may be expressed as recombinants and used as immunogens.

Initial studies on the effect of mutations in the N- and C-helical regions of gp41 on envelope structure and function were carried out using synthetic peptides modeling these domains. The N-helical region, which by sequence analysis predicts a coiled-coil structure, is among the most conserved in the envelope protein and is distinguished by strict primary sequence requirements. The coiled-coil motif is characterized by a 4-3 spacing (heptad repeat) of hydrophobic amino acid residues, most often leucine or isoleucine. The regular repeat of these residues has resulted in the term “leucine zipper” to describe coiled-coil domains. Substitution of these invariant residues usually results in a dramatic decrease or complete loss of the coiled coil structure as demonstrated on the N-helical gp41 region by substituting a proline residue for an isoleucine at position 578. This single change resulted in a complete loss of structure as measured by circular dichroism (Wild, C., etal., Proc. Natl. Acad. Sci. USA 89:10537-10541 (1992)). In addition, point mutations within the N-helical domain have dramatic effects on both structure and function, but do not interfere with the expression of envelope protein (Wild, C., Proc. Natl. Acad. Sci. USA 91:12676-12680 (1994)).

The C-helix of gp41 has been similarly characterized. Like the N-helix, the primary amino acid sequence of the C-helix is predictive of α-helical secondary structure. However, unlike its N-terminal counterpart, when modeled as a synthetic peptide, the C-helix does not exhibit stable solution structure. It is widely believed that the inability of peptides to model the structural components of this gp41 domain are due in part to its amphipathic nature. In the absence of an appropriate interface, i.e., the surface provided by the super-helical grove of the N-terminal coiled coil, the stabilization provided by the interaction of the regularly placed hydrophobic and hydrophilic amino acid residues with like surfaces is not realized and secondary structure does not form. While this region of gp41 exists as an α-helix in the context of the six-helix bundle, the structure assumed by the isolated form of this entry determinant remains unknown. However, it is believed that the combination of amphipathic nature and proximity to a hydrophobic surface (the infected cell or viral membrane) favors the formation of an extended α-helical conformation most likely positioned along the interface provided by the external environment and the viral membrane. The effect of mutations in this region of the gp41 on both envelope expression and function have been determined (Salzwedel, K., et al., J. Virol. 73:2469-2480 (1999)).

The structure-disrupting mutations in the N-helical coiled-coil region will result in the generation of envelope expressing stable fusion-active C-helical determinants. Conversely, the structure-disrupting mutations in the C-helical domain give rise to envelope presenting stable isolated forms of the N-helical coiled coil. In each case, the stabilized forms of fusion-active envelope proteins may be used as vaccine immunogens.

Structure-disrupting mutations effective in gp41 sequences from the HIV-1_{LAI} isolate would be expected to be effective in other systems such as SF162 due to the high degree of sequence homology in the N- and C-helical regions of the transmembrane protein. For example, C-helical regions of the HXB2 and SF162 (isolates of HIV-1) transmembrane proteins exhibit near complete sequence homology (YTSLIHSLIEESQNQQEKNEQELLELDKWASLWNWF (HXB2) (SEQ ID NO:85) and YT--------LIEESQNQQEKNEQELLELDKWASLWNWF (SF162) (SEQ ID NO:86)). The single difference in the N-helix is conserved (V to I) as are the two differences in the C-helix. Due to this high degree of similarity, the results generated in one transmembrane protein will likely readily apply to the others.

Multiple mutations in the N-helical domain can occur at, for example, amino acid positions 571, 578 and/or 585 of gp41. Several studies have established that appropriate changes at these invariant residues will result in the loss of α-helical secondary structure (Wild, C., et al., Proc. Natl. Acad. Sci. USA 89:10537-10541 (1992); Wild, C., Proc. Natl. Acad. Sci. USA 91:12676-12680 (1994)). Shown below is the sequence for residues 558-595 (SEQ ID NO:7) of the HIV-1_{LAI} gp41 protein. The a and d subscripts denote the 4-3 positions of the heptad repeat.

```
N N L L R A I E A Q Q H L L Q L T V W G I K Q L Q A R I L A V E R Y L K D Q
        d a d a d a d a d a
                                                  571 578 585
```

The following mutations in the gp41 sequence may be made:

1) 578 Isoleucine to Proline, -p1 2) 571 Leucine to Glycine, 578 Isoleucine to Proline or

3) 571 Leucine to Glycine, 578 Isoleucine to Proline, 585 Isoleucine to Glycine.

These point mutations introduced into each of the recombinant forms of gp41 result in the loss of secondary structure in the N-helical domain. Synthetic peptides containing these changes may be prepared and characterized by circular dichroism for a-helical structure (Wild, C., et al., Proc. Natl. Acad. Sci. USA 89:10537-10541 (1992); Wild, C., Proc. Natl. Acad. Sci. USA 91:12676-12680 (1994)). These sequences, deficient in secondary structure, may be incorporated into a protein expression system, tested for expression level in the relevant system and analyzed for disruption of six-helix bundle formation by lysate and surface immunoprecipitation experiments using polyclonal sera generated against this complex structure.

A similar approach may be taken to generate gp41 peptides deficient for structure in the C-helical domain. Shown below is the amino acid sequence for residues 643-678 (SEQ ID NO:1) of the HIV-1_{LAI}gp41 protein.

Y T S L I H S L I E E S Q N Q Q E K N E Q E L L E L D K W A S L W N W F
d a d a d a d a d a
647 654 661

Possible mutations in the gp41 sequence include:

1) 654 Serine to Glycine,

2) 647 Isoleucine to Glycine, 654 Serine to Glycine, or

3) 647 Isoleucine to Glycine, 654 Serine to Glycine, 661 Asparagine to Glycine.

Unlike the N-helix, when modeled as a peptide, the C-helical region of gp41 is not structured. However, when mixed with the N-peptide, the C-peptide does takes on a-helical structure as part of the core structure complex. The structure forms in vitro on mixing the peptides and can be characterized spectrophotometrically (Lu, M., et al., Nat. Struct. Biol. 2:1075-1082 (1995)). The initial determination of the effect of the mutations on C-helix structure may be performed by analyzing the ability of the mutant C-peptide to interact with the N-peptide and form the six-helix bundle. This analysis may be carried out using circular dichroism as set forth in Example 13. As proposed above for the N-helical mutants, each of the C-peptide sequences shown to be deficient for structure may be incorporated into a protein expression system, tested for level of expression and analyzed for effect on six-helix bundle formation by surface immunoprecipitation assays prior to expression.

### Vaccine Applications

Vaccine delivery vehicles may include adjuvants, liposomes, microparticles, pseudovirions and other methods of introducing proteins. In addition, the vaccines of the present invention may be employed in such forms as capsules, liquid solutions, suspensions or elixirs for oral administration, or sterile liquid forms such as solutions or suspensions. Any inert carrier is preferably used, such as saline, phosphate-buffered saline, or any such carrier in which the conjugate vaccine has suitable solubility properties. The vaccines may be in the form of single dose preparations or in multi-dose flasks which can be used for mass vaccination programs. Reference is made to Remington's Pharmaceutical Sciences, Osol, ed., Mack Publishing Co., Easton, Pa. (1980), and New Trends and Developments in Vaccines, Voller, et al., eds., University Park Press, Baltimore, Md. (1978), for methods of preparing and using vaccines.

The vaccine immunogens of the present invention may further comprise adjuvants which enhance production of HIV-specific antibodies. Such adjuvants include, but are not limited to, various oil formulations such as Freund's complete adjuvant (CFA), the Ribi adjuvant system (RAS), MF59, stearyl tyrosine (ST, see U.S. Pat. No. 4,258,029), the dipeptide known as MDP, saponins and saponin derivatives such as Quil A and QS-21, aluminum hydroxide and lymphatic cytokine. Preferably, an adjuvant will aid in maintaining the secondary and quaternary structure of the immunogens. Adjuvant formulations which have been developed specifically for subunit applications or to preserve and present native protein conformations may also be used. MF59, a squalene/water emulsion produced by Chiron Corp., is an example of such an adjuvant. MF59 has been shown to result in an elevated humoral immune response to subunit antigens (Ott, G., et al., Vaccine 13:1557-1562 (1995); Cataldo, D. M. and Van Nest, G., Vaccine 15:1710-1715 (1997)). Importantly, this adjuvant has exhibited favorable compatibility in studies involving humans.

Freund's adjuvant is an emulsion of mineral oil and water which is mixed with the immunogenic substance. Although Freund's adjuvant is powerful, it is usually not administered to humans. Instead, the adjuvant alum (aluminum hydroxide) or ST may be used for administration to a human. The vaccine may be absorbed onto the aluminum hydroxide from which it is slowly released after injection. The vaccine may also be encapsulated within liposomes according to Fullerton, U.S. Pat. No. 4,235,877, or mixed with liposomes or lipid mixtures to provide an environment similar to the cell surface environment.

There is evidence that traditional formulations, such as Freund's adjuvant (both complete and incomplete) and Alum gel at least partially denature antigen resulting in the destruction or under-representation of conformational epitopes. The Ribi adjuvant system (RAS), which belongs to the monophosphoryl-lipid A (MPL) containing-adjuvants, may be used to overcome this problem. Results from several studies indicate that antigen formulated using MPL-containing adjuvants elicited antibodies that preferentially bound native rather than denatured antigen (Earl, P. L., et al., J. Virol 68:3015-3026 (1994); VanCott T. C., et al., J. Virol 71:4319-4330 (1997)).

Carrier molecules can also be used to enhance the neutralizing antibody response to immunogens modeling early fusion-active structures. A significant body of work illustrates that coupling small molecules to large proteins results in an enhanced immune response. This enhancement is believed to be due to several factors including T-cell help (provided by T helper epitopes contained within the carrier proteins), more native-like presentation of the antigen in the context of a large molecule and a general increase in immune recognition of the large molecule conjugate.

The traditional carrier molecule keyhole limpet hemocyanin (KLH) can be employed to give the peptides the freedom to assume the appropriate and necessary conformation(s) following conjugation. Thus, each antigen can be prepared with an N-terminal cystine residue and coupled to a carrier through the sulfhydryl group of the terminal residue. Immunogens can then be coupled to KLH through the sulfhydryl group of the N-terminal cysteine residue.

In a preferred embodiment, the present invention relates to methods of inducing an immune response in an animal comprising administering to the animal, the vaccine immunogen of the invention in an amount effective to induce an immune response. Optionally, the vaccine immunogen may be coadministered with effective amounts of other immunogens to generate multiple immune responses in the animal.

In preferred aspects of the invention, the vaccine immunogens can be employed to immunize an HIV-1 infected individual such that levels of HIV-1 will be reduced in the individual. In another aspect, the vaccine immunogens can be employed to immunize a non-HIV-1 infected individual so that, following a subsequent exposure to HIV-1 that would normally result in HIV-1 infection, the level of HIV-1 will be non-detectable using current diagnostic tests.

In alternative embodiments, the vaccine immunogens can be used to raise antibodies by methods known to those of ordinary skill in the art. The antibodies raised can then be administered to an HIV-1 infected or non-HIV-1 infected individual. If administered to an HIV-1 infected individual, then the antibodies should be administered such that levels of HIV-1 will be reduced in the individual. If administered to a non-HIV-1 infected individual, then the antibodies should be administered such that following a subsequent exposure to HIV-1 that would normally result in HIV-1 infection, the level of HIV-1 will be non-detectable using current diagnostic tests.

Antiviral activity of neutralizing antibodies generated by the immunization with vaccine immunogens can be evaluated in both cell-cell fusion and neutralization assays. In the latter assay, a representative sample of lab adapted and primary virus isolates is used. Both assays are carried out according to known protocols as described in, for example, Wild, C., et al., Proc. Natl. Acad. Sci. USA 89:10537-10541 (1992), Wild, C., et al., Proc. Natl. Acad. Sci. USA 91:12676-12680 (1994), and Wild, C., et al., Proc. Natl. Acad. Sci. USA 91:9770-9774 (1994).

For hybridoma production, samples can be screened by a number of techniques to characterize binding to fusion-active epitopes. One approach involves ELISA binding to the inventive immunogens. Animals with sera samples which test positive for binding to one or more of the fusion-active immunogens are candidates for use in MAb production. The criteria for selection of animals to be used in MAb production is based on the evidence of neutralizing antibody in the animals'sera or in the absence of neutralization, appropriate binding patterns against fusion-active immunogens.

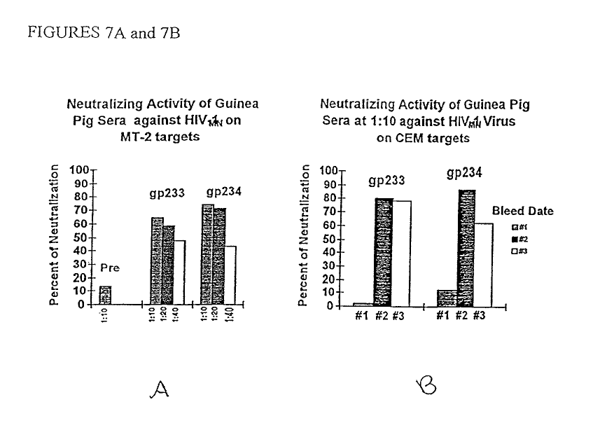

In the neutralization assay, test sera can be incubated at a 1:10 dilution with virus, e.g., HIV-1IIIB for 1 hour at 37° C. At the end of this time, target cells can be added (CEM) and the experiment returned to the incubator. On days 1, 3 and 5, post-infection complete media changes can be carried out. On day 7, PI culture supernatant can be harvested. Levels of virus replication can then be determined by p24 antigen capture. Levels of replication in test wells can be normalized to virus only controls. See FIGS. 7A and 7B.

Hybridoma supernatants derived from MAb production may be screened for ELISA, lysate and surface immunoprecipitation assays for binding to fusion-active forms of envelope. Samples which are positive in any of the binding assays may be screened for their ability to neutralize a panel of HIV-1 isolates as described above. These isolates include lab adapted and primary virus strains, syncytium- and non-syncytium-inducing isolates, virus representing various geographic subtypes and viral isolates which make use of the range of second receptors during virus entry. The neutralization assays employ either primary cell or cell line targets as required.

The following assays are examples of assays used to assess whether immunogens of the invention are fusion-active:

### ELISA Assay

Nunc Immulon 2 HB plates are coated with 1 μg/well of peptide. Approximately, 100 μl of sample at desired dilution are added in duplicate and allowed to incubate for 2 hours at 37° C. Hybridoma supernatants are tested neat while polyclonal sera are assayed at an initial concentration of 1:100 followed by 4-fold dilutions. Following incubation, samples are removed and plates are washed with PBS+0.05% Tween-20, and 100 μl/well of diluted phosphatase-labeled secondary antibody (Sigma) is added. The secondary antibody-conjugate is diluted in blocking buffer to a final concentration of 1:1500 and added. Following incubation at room temperature, plates are washed and substrate (Sigma fast p-nitrophenyl phosphate) is added. Following development, plates are read at 405 nm.

### Western Blot Analysis

Commercial HIV-1 western blot strips are pre-wet with wash buffer (PBS+0.05% Tween-20). Samples are diluted in buffer (PBS, 0.05% Tween-20, 5% evaporated milk) to a final concentration of 1:5 for hybridoma supernatants and 1:200 for polyclonal sera and added to the strips. Following incubation (2 hours with rocking), the strips are washed (3×5 min intervals) with wash buffer. Peroxidase-labeled secondary antibody (Kirkgaard & Perry Laboratories) is added at a concentration of 1:5000 and incubated with rocking for 1 hour. Strips are washed again as described previously and TMB substrate is added. Color development is stopped by the addition of water.

### Lysate Immunoprecipitation Assay

Hybridoma supernatants or immunosera are incubated overnight at 4° C. in 200 μl PBS containing 4.2 μl of HIV-1 IIIB cell lysate. The lysate is prepared from acute infection of the H9 cell line. Immune complexes are precipitated by the addition of protein A and G Agarose, washed and analyzed by 10% SDS-PAGE (NOVEX), transferred to nitrocellulose and immunoblotted with anti-gp4 monoclonal antibody Chessie 8 (obtained from NIH AIDS Research and Reference Reagent Program), and detected by chemiluminescence (Amersham) and autoradiography.

### gp41 α-Helical Peptides (N-Helix or C-Helix)

Peptides useful in the present invention are gp41 α-helical peptides which are defined by their ability to disrupt the formation of one or more structural intermediates necessary for viral fusion and entry by interacting with a region complementary to the peptide on the viral envelope protein. The peptides may be synthesized or prepared by techniques well-known in the art. See, e.g., Creighton, Proteins: Structures and Molecular Principles, W. H. Freeman & Co., New York, N.Y. (1983), which is incorporated herein by reference in its entirety. Peptides, for example, can be synthesized as a solid support or in solution or made using recombinant DNA techniques wherein the nucleotide sequences encoding the peptides may be synthesized and/or cloned, and expressed according to techniques well-known to those of ordinary skill in the art. See, e.g., Sambrook, et al., Molecular Cloning, A Laboratory Manual, vols. 1-3, Cold Spring Harbor Press, Cold Spring Harbor, N.Y. (1989).

The peptides employed in the present invention may alternatively be synthesized such that one or more of the bonds which link the amino acid residues of the peptides are non-peptide bonds. These alternative non-peptide bonds may be formed by utilizing reactions well-known to those in the art, and may include, but are not limited to, imino, ester, hydrazide, semicarbazide, and azo bonds. In yet another embodiment of the invention, peptides comprising the sequences described below may be synthesized with additional chemical groups present at their amino and/or carboxy termini, such that, for example, the stability, bioavailability and/or disruptive activity of the peptides is enhanced. For example, hydrophobic groups such as carbobenzoxyl, dansyl, ort-butyloxycarbonyl groups, may be added to the peptide's amino termini. Likewise, an acetyl group or a 9-fluorenylmethoxy-carbonyl group may be placed at the peptide's amino termini. Additionally, a hydrophobic group such as t-butyloxycarbonyl or an amido group may be added to the peptide's carboxy termini.

Further, the peptides of the invention may be synthesized such that their steric configuration is altered. For example, the D-isomer of one or more of the amino acid residues of the peptide may be used, rather than the usual L-isomer. Still further, at least one of the amino acid residues of the peptides may be substituted by one of the well-known non-naturally occurring amino acid residues. Alterations such as these may serve to increase the stability, bioavailability and/or inhibitory action of the peptides.

Any of the peptides may additionally, have a non-peptide macromolecular carrier group covalently attached to their amino and/or carboxy termini. Such macromolecular carrier groups may include, for example, lipid-fatty acid conjugates, polyethylene glycol, or carbohydrates.

Peptides are defined herein as organic compounds comprising two or more amino acids covalently joined by peptide bonds. Peptides may be referred to with respect to the number of constituent amino acids, i.e., a dipeptide contains two amino acid residues, a tripeptide contains three amino acid residues, etc. Peptides containing ten or fewer amino acids may be referred to as oligopeptides, while those with more than ten amino acid residues may be referred to as polypeptides.

Peptide sequences defined herein are represented by one-letter symbols for amino acid residues as follows:

A alanine

R arginine

N asparagine

D aspartic acid

C cysteine

Q glutamine

E glutamic acid

G glycine

H histidine

I isoleucine

L leucine

K lysine

M methionine

F phenylalanine

P proline

S serine

T threonine

W tryptophan

Y tyrosine

V valine

Useful gp41 α-helical (N-helix and C-helix) peptides are the selected from the group consisting of: a peptide comprising SEQ ID NO:1, a peptide comprising a fragment of SEQ ID NO:1, a peptide comprising SEQ ID NO:2, a peptide comprising a fragment of SEQ ID NO:2, a peptide comprising SEQ ID NO:3, a peptide comprising a fragment of SEQ ID NO:3, a peptide comprising SEQ ID NO:4, a peptide comprising a fragment of SEQ ID NO:4, a peptide comprising SEQ ID NO:5, a peptide comprising a fragment of SEQ ID NO:5, a peptide comprising SEQ ID NO:6, a peptide comprising a fragment of SEQ ID NO:6, a peptide comprising SEQ ID NO:7, a peptide comprising a fragment of SEQ ID NO:7, a peptide comprising SEQ ID NO:9, a peptide comprising a fragment of SEQ ID NO:9, a peptide comprising any combination of SEQ ID NOS:1-7 and 9, a peptide comprising any combination of fragments of SEQ ID NOS:1-7 and 9, a peptide functionally equivalent to any one of SEQ ID NOS:1-7 and 9, a homolog of any of SEQ ID NOS:1-7 and 9, an analog of any of SEQ ID NOS:1-7 and 9, influenza hemagglutinin epitope, an epitope-tagged peptide and mixtures thereof.

### C-Helical Peptides

The C-terminal helix region of HIV-1 gp41 has the amino acid sequence: WNNMTWMEWDREINNYTSLIHSLIEESQNQQEKNEQELLELDKWASL WNWFNITNW (SEQ ID NO:13)

The peptides of the invention may include peptides comprising SEQ ID NO:13 with or without amino acid insertions which consist of single amino acid residues or stretches of residues ranging from 2 to 15 amino acids in length. One or more insertions may be introduced into the peptide, peptide fragment, analog and/or homolog.

The peptides of the invention may include peptides comprising SEQ ID NO:13 with or without amino acid deletions of the full length peptide, analog, and/or homolog. Such deletions consist of the removal of one or more amino acids from the full-length peptide sequence, with the lower limit length of the resulting peptide sequence being 4 to 6 amino acids. Such deletions may involve a single contiguous portion or greater than one discrete portion of the peptide sequences.

Examples of C-helical Domain Peptide Sequences (all sequences are listed from N-terminus to C-terminus) from different HIV strains include, but are not limited to, the following:

| | |
|---|---|
| HIV-1 Group M: Subtype B Isolate: LAI | |
| WNNMTWMEWDREINNYTSLIHSLIEESQNQQEKNEQELLELDKWASL | (SEQ ID NO:13) |
| WNWFNITNW | |
| WMEWDREINNYTSLIHSLIEESQNQQEKNEQELLELDKWASLWNWF | (SEQ ID NO:15) |
| P-16 WMEWDREINNYTSLIHSLIEESQNQQEKNEQELL | (SEQ ID NO:16) |
| P-18 YTSLIHSLIEESQNQQEKNEQELLELDKWASLWNWF | (SEQ ID NO:1) |
| Subtype B Isolate: ADA | |
| WMEWEREIENYTGLIYTLIEESQNQQEKNEQDLLALDKWASLWNWF | (SEQ ID NO:17) |
| WMEWEREIENYTGLIYTLIEESQNQQEKNEQDLL | (SEQ ID NO:18) |
| YTGLIYTLIEESQNQQEKNEQDLLALDKWASLWNWF | (SEQ ID NO:19) |
| Subtype B Isolate: JRFL | |
| WMEWEREIDNYTSEIYTLIEESQNQQEKNEQELLELDKWASLWNWF | (SEQ ID NO:20) |
| WMEWEREIDNYTSEIYTLIEESQNQQEKNEQELL | (SEQ ID NO:21) |
| YTSEIYTLIEESQNQQEKNEQELLELDKWASLWNWF | (SEQ ID NO:22) |
| Subtype B Isolate: 89.6 | |
| WMEWEREIDNYTDYIYDLLEKSQTQQEKNEKELLELDKWASLWNWF | (SEQ ID NO:23) |
| WMEWEREIDNYTDYIYDLLEKSQTQQEKNEKELL | (SEQ ID NO:24) |
| YTDYIYDLLEKSQTQQEKNEKELLELDKWASLWNWF | (SEQ ID NO:25) |
| Subtype C Isolate: BU910812 | |
| WIQWDREISNYTGIIYRLLEESQNQQENNEKDLLALDKWQNLWSWF | (SEQ ID NO:26) |
| WIQWDREISNYTGIIYRLLEESQNQQENNEKDLL | (SEQ ID NO:27) |
| YTGIIYRLLEESQNQQENNEKDLLALDKWQNLWSWF | (SEQ ID NO:28) |
| Subtype D Isolate: 92UG024D | |
| WMEWEREISNYTGLIYDLIEESQIQQEKNEKDLLELDKWASLWNWF | (SEQ ID NO:29) |
| WMEWEREISNYTGLIYDLIEESQIQQEKNEKDLL | (SEQ ID NO:30) |
| YTGLIYDLIEESQIQQEKNEKDLLELDKWASLWNWF | (SEQ ID NO:31) |
| Subtype F Isolate: BZ163A | |
| WMEWQKEISNYSNEVYRLIEKSQNQQEKNEQGLLALDKWASLWNWF | (SEQ ID NO:32) |
| WMEWQKEISNYSNEVYRLIEKSQNQQEKNEQGLL | (SEQ ID NO:33) |
| YSNEVYRLIEKSQNQQEKNEQGLLALDKWASLWNWF | (SEQ ID NO:34) |
| Subtype G Isolate: FI.HH8793 | |
| WIQWDREISNYTQQIYSLIEESQNQQEKNEQDLLALDNWASLWTWF | (SEQ ID NO:35) |
| WIQWDREISNYTQQIYSLIEESQNQQEKNEQDLL | (SEQ ID NO:36) |
| YTQQIYSLIEESQNQQEKNEQDLLALDNWASLWTWF | (SEQ ID NO:37) |
| Subtype H Isolate: BE.V1997 | |
| WMEWDRQIDNYTEVIYRLLELSQTQQEQNEQDLLALDKWDSLWNWF | (SEQ ID NO:38) |
| WMEWDRQIDNYTEVIYRLLELSQTQQEQNEQDLL | (SEQ ID NO:39) |
| YTEVIYRLLELSQTQQEQNEQDLLALDKWDSLWNWF | (SEQ ID NO:40) |
| Subtype J Isolate: SE.SE92809 | |
| WIQWEREINNYTGIIYSLIEEAQNQQENNEKDLLALDKWTNLWNWFN | (SEQ ID NO:41) |
| WIQWEREINNYTGIIYSLIEEAQNQQENNEKDLL | (SEQ ID NO:42) |
| YTGIIYSLIEEAQNQQENNEKDLLALDKWTNLWNWFN | (SEQ ID NO:43) |
| Group N Isolate: CM.YBF30 | |
| WQQWDEKVRNYSGVIFGLIEQAQEQQNTNEKSLLELDQWDSLWSWF | (SEQ ID NO:44) |
| WQQWDEKVRNYSGVIFGLIEQAQEQQNTNEKSLL | (SEQ ID NO:45) |
| YSGVIFGLIEQAQEQQNTNEKSLLELDQWDSLWSWF | (SEQ ID NO:46) |
| Group O Isolate: CM.ANT70C | |
| WQEWDRQISNISSTIYEEIQKAQVQQEQNEKKLLELDEWASIWNWL | (SEQ ID NO:47) |
| WQEWDRQISNISSTIYEEIQKAQVQQEQNEKKLL | (SEQ ID NO:48) |
| ISSTIYEEIQKAQVQQEQNEKKLLELDEWASIWNWL | (SEQ ID NO:49) |

Stabilizing peptides may include the C-helical peptide P-18 which corresponds to amino acid residues 638 to 673 of the transmembrane protein gp41 from the HIV-1_{LAI} isolate, and has the 36 amino acid sequence (reading from amino to carboxy terminus):

NH₂ -YTSLIHSLIEESQNQQEKNEQELLELDKWASLWNWF-COOH (SEQ ID NO:1)

In addition to the full-length P-18 36-mer (SEQ ID NO:1), the peptides of the invention may include truncations of the C-helical peptides which exhibit stabilizing activity. Such truncated peptides may comprise peptides of between 3 and 36 amino acid residues, i.e., peptides ranging in size from a tripeptide to a 36-mer polypeptide, and may include, but are not limited to, those listed in Tables I and II, below. Peptide sequences in these tables are listed from amino (left) to carboxy (right) terminus. “X” may represent an amino group (—NH₂) and “Z” may represent a carboxyl (—COOH) group. Alternatively, as described below, “X” and/or “Z” may represent a hydrophobic group, an acetyl group, a FMOC group, an amido group, or a covalently attached macromolecule.

TABLE I
Carboxy Truncations of SEQ ID NO:1

X-YTS-Z
X-YTSL-Z
X-YTSLI-Z
X-YTSLIH-Z
X-YTSLIHS-Z
X-YTSLIHSL-Z
X-YTSLIHSLI-Z
X-YTSLIHSLIE-Z
X-YTSLIHSLIEE-Z
X-YTSLIHSLIEES-Z
X-YTSLIHSLIEESQ-Z
X-YTSLIHSLIEESQN-Z
X-YTSLIHSLIEESQNQ-Z
X-YTSLIHSLIEESQNQQ-Z
X-YTSLIHSLIEESQNQQE-Z
X-YTSLIHSLIEESQNQQEK-Z
X-YTSLIHSLIEESQNQQEKN-Z
X-YTSLIHSLIEESQNQQEKNE-Z
X-YTSLIHSLIEESQNQQEKNEQ-Z
X-YTSLIHSLIEESQNQQEKNEQE-Z
X-YTSLIHSLIEESQNQQEKNEQEL-Z
X-YTSLIHSLIEESQNQQEKNEQELL-Z
X-YTSLIHSLIEESQNQQEKNEQELLE-Z
X-YTSLIHSLIEESQNQQEKNEQELLEL-Z
X-YTSLIHSLIEESQNQQEKNEQELLELD-Z
X-YTSLIHSLIEESQNQQEKNEQELLELDK-Z
X-YTSLIHSLIEESQNQQEKNEQELLELDKW-Z
X-YTSLIHSLIEESQNQQEKNEQELLELDKWA-Z
X-YTSLIHSLIEESQNQQEKNEQELLELDKWAS-Z
X-YTSLIHSLIEESQNQQEKNEQELLELDKWASL-Z
X-YTSLIHSLIEESQNQQEKNEQELLELDKWASLW-Z
X-YTSLIHSLIEESQNQQEKNEQELLELDKWASLWN-Z
X-YTSLIHSLIEESQNQQEKNEQELLELDKWASLWNW- Z
X-YTSLIHSLIEESQNQQEKNEQELLELDKWASLWNWF- Z
The one letter amino acid code is used.
“X” may represent a hydrogen attached to the terminal amino group, an amino protecting group including, but not limited to, carbobenzoxyl, dansyl, or t-butyloxycarbonyl; an acetyl group; a 9-fluorenylmethoxy-carbonyl (FMOC) group; a macromolecular carrier group including, but not limited to, lipid-fatty acid conjugates, polyethylene glycol, or carbohydrates.
“Z” may represent a terminal carboxyl (COOH); an amido group; an ester group (COOR) including, but not limited to, a t-butyloxycarbonyl group; a macromolecular carrier group including, but not limited to, lipid-fatty acid conjugates, polyethylene glycol, or carbohydrates.

TABLE II
Amino Truncations of SEQ ID NO:1

X-NWF-Z
X-WNWF-Z
X-LWNWF-Z
X-SLWNWF-Z
X-ASLWNWF-Z
X-WASLWNWF-Z
X-KWASLWNWF-Z
X-DKWASLWNWF-Z
X-LDKWASLWNWF-Z
X-ELDKWASLWNWF-Z
X-LELDKWASLWNWF-Z
X-LLELDKWASLWNWF-Z
X-ELLELDKWASLWNWF-Z
X-QELLELDKWASLWNWF-Z
X-EQELLELDKWASLWNWF-Z
X-NEQELLELDKWASLWNWF-Z
X-KNEQELLELDKWASLWNWF-Z
X-EKNEQELLELDKWASLWNWF-Z
X-QEKNEQELLELDKWASLWNWF-Z
X-QQEKNEQELLELDKWASLWNWF-Z
X-NQQEKNEQELLELDKWASLWNWF-Z
X-QNQQEKNEQELLELDKWASLWNWF-Z
X-SQNQQEKNEQELLELDKWASLWNWF-Z
X-ESQNQQEKNEQELLELDKWASLWNWF-Z
X-EESQNQQEKNEQELLELDKWASLWNWF-Z
X-IEESQNQQEKNEQELLELDKWASLWNWF-Z
X-LIEESQNQQEKNEQELLELDKWASLWNWF-Z
X-SLIEESQNQQEKNEQELLELDKWASLWNWF-Z
X-HSLIEESQNQQEKNEQELLELDKWASLWNWF-Z
X-IHSLIEESQNQQEKNEQELLELDKWASLWNWP-Z
X-LIHSLIEESQNQQEKNEQELLELDKWASLWNWF-Z
X-SLIHSLIEESQNQQEKNEQELLELDKWASLWNWF-Z
X-TSLIHSLIEESQNQQEKNEQELLELDKWASLWNWF-Z
X-YTSLIHSLIEESQNQQEKNEQELLELDKWASLWNWF-Z
The one letter amino acid code is used.
“X” may represent a hydrogen attached to the terminal amino group, an amino protecting group including, but not limited to, carbobenzoxyl, dansyl, or t-butyloxycarbonyl; an acetyl group; a 9-fluorenylmethoxy-carbonyl (FMOC) group; a macromolecular carrier group including, but not limited to, lipid-fatty acid conjugates, polyethylene glycol, or carbohydrates.
“Z” may represent a terminal carboxyl (COOH); an amido group; an ester group (COOR) including, but not limited to, a t-butyloxycarbonyl group; amacromolecular carrier group including, but not limited to, lipid-fatty acid conjugates, polyethylene glycol, or carbohydrates.

The stabilizing peptides also include analogs of P-18 and/or P-18 truncations which may include, but are not limited to, peptides comprising the P-18 sequence (SEQ ID NO:1), or a P-18 truncated sequence, containing one or more amino acid substitutions, insertions and/or deletions. Analogs of P-18 homologs are also within the scope of the invention. The P-18 analogs exhibit disruptive activity, and may possess additional advantageous features, such as, for example, increased bioavailability and/or stability.

Amino acid substitutions may be of a conserved or non-conserved nature. Conserved amino acid substitutions consist of replacing one or more amino acids of the P-18 (SEQ ID NO:1) peptide sequence with amino acids of similar charge, size and/or hydrophobicity characteristics, such as, for example, a glutamic acid (E) to aspartic acid (D) amino acid substitution. Non-conserved substitutions consist of replacing one or more amino acids of the P-18 (SEQ ID NO:1) peptide sequence with amino acids possessing dissimilar charge, size and/or hydrophobicity characteristics, such as, for example, a glutamic acid (E) to valine (V) substitution.

Amino acid insertions may consist of single amino acid residues or stretches of residues ranging from 2 to 15 amino acids in length. The insertions may be made at the carboxy or amino terminal end of the P-18 or P-18 truncated peptide, as well as at a position internal to the peptide. It is contemplated that insertions made at either the carboxy or amino terminus of the peptide of interest may be of a broader size range, with about 2 to about 50 amino acids being preferred. One or more insertions may be introduced into P-18 (SEQ ID NO:1), P-18 fragments, P-18 analogs and/or P-18 homologs.

Preferred amino or carboxy terminal insertions are peptides ranging from about 2 to about 50 amino acid residues in length, corresponding to gp41 protein regions either amino to or carboxy to the actual P-18 gp41 amino acid sequence, respectively. Thus, a preferred amino terminal or carboxy terminal amino acid insertion would contain gp41 amino acid sequences found immediately amino to or carboxy to the P-18 region of the gp41 protein.

Deletions from P-18 (SEQ ID NO:1), P-18 truncations, P-18 fragments, P-18 analogs and/or P-18 homologs are also within the scope of the invention. Such deletions consist of the removal of one or more amino acids from any of the P-18 peptide sequences, with the lower limit length of the resulting peptide sequence being 4 to 6 amino acids. Such deletions may involve a single contiguous portion of a peptide sequence or greater than one discrete portion of a peptide sequence.

The peptides may further include homologs of P-18 (SEQ ID NO:1) and P-18 truncations which exhibit disruptive activity. Such P-18 homologs are peptides whose amino acid sequences are comprised of the amino acid sequences of peptide regions of other, i.e., other than HIV-1_{LAI}, viruses that correspond to the gp41 peptide region from which P-18 (SEQ ID NO:1) was derived. Such viruses may include, but are not limited to, other HV-1 isolates and HIV-2 isolates. P-18 homologs derived from the corresponding gp41 peptide region of other HIV-1 isolates, i.e., non-HIV-1_{LAI}, may include, for example, peptide sequences as shown below.

| | |
|---|---|
| NH₂-YTNTIYTLLEESQNQQEKNEQELLELDKWASLWNWF-COOH | (SEQ ID NO:2); |
| NH₂-YTGIIYNLLEESQNQQEKNEQELLELDKWANLWNWF-COOH | (SEQ ID NO:3); and |
| NH₂-YTSLIYSLLEKSQIQQEKNEQELLELDKWASLWNWF-COOH | (SEQ ID NO:4). |

SEQ ID NO:2, SEQ ID NO:3 and SEQ ID NO:4 are derived from HIV-1_{SF2}, IIIV-1_{RF}, and IIIV-1_{MN} isolates, respectively. The P-18 homologs may also include truncations, amino acid substitutions, insertions and/or deletions, as described above.

In addition, peptides derived from HIV-2 isolates can be employed as stabilizing peptides. A useful peptide derived from the HIV-2_{NHZ} isolate has the 36 amino acid sequence (reading from amino to carboxy terminus):

NH₂-LEANISQSLEQAQIQQEKNMYELQKLNSWDVFTNWL-COOH (SEQ ID NO:5)

Tables III and IV show truncations of the HIV-2_{NHZ}P-18 homolog, which may comprise peptides of between 3 and 36 amino acid residues, i.e., peptides ranging in size from a tripeptide to a 36-mer polypeptide. Peptide sequences in these tables are listed from amino (left) to carboxy (right) terminus. “X” may represent an amino group (—NH₂) and “Z” may represent a carboxyl (—COOH) group. Alternatively, as described below, “X” and/or “Z” may represent a hydrophobic group, an acetyl group, a FMOC group, an amido group, or a covalently attached macromolecule.

TABLE III
Carboxy Truncations of HIV-2_{NIHZ} Peptide (SEQ ID NO:5)

X-LEA-Z
X-LEAN-Z
X-LEANI-Z
X-LEANIS-Z
X-LEANISQ-Z
X-LEANISQS-Z
X-LEANISQSL-Z
X-LEANISQSLE-Z
X-LEANISQSLEQ-Z
X-LEANTSQSLEQA-Z
X-LEANISQSLEQAQ-Z
X-LEANISQSLEQAQI-Z
X-LEANISQSLEQAQIQ-Z
X-LEANISQSLEQAQIQQ-Z
X-LEANISQSLEQAQIQQE-Z
X-LEANISQSLEQAQIQQEK-Z
X-LEANISQSLEQAQIQQEKN-Z
X-LEANISQSLEQAQIQQEKNM-Z
X-LEAMISQSLEQAQIQQEKNMY-Z
X-LEANISQSLEQAQIQQEKNMYE-Z
X-LEANISQSLEQAQIQQEKNMYEL-Z
X-LEANISQSLEQAQIQQEKNMYELQ-Z
X-LEANISQSLEQAQIQQEKNMYELQK-Z
X-LEANISQSLEQAQIQQEKNMYELQKL-Z
X-LEANISQSLEQAQIQQEKNMYELQKLN-Z
X-LEANISQSLEQAQIQQEKNMYELQKLNS-Z
X-LEANISQSLEQAQIQQEKNMYELQKLNSW-Z
X-LEANISQSLEQAQIQQEKNMYELQKLNSWD-Z
X-LEANISQSLEQAQIQQEKNNYELQKLNSWDV-Z
X-LEANISQSLEQAQIQQEKNMYELQKLNSWDVF-Z
X-LEANISQSLEQAQIQQEKNNYELQKLNSWDVFT-Z
X-LEANISQSLEQAQIQQEKNMYELQKLNSWDVFTN-Z
X-LEANISQSLEQAQIQQEKNNYELQKLNSWDVFTNW-Z
X-LEANISQSLEQAQIQQEKNNYELQKLNSWDVFTNWL-Z
The one letter amino acid code is used.
“X” may represent a hydrogen attached to the terminal amino group, an amino protecting group including, but not limited to, carbobenzoxyl, dansyl, or t-butyloxycarbonyl; an acetyl group; a 9-fluorenylmethoxy-carboflyl (EMOC) group; a macromolecular carrier group including, but not limited to, lipid-fatty acid conjugates, polyethylene glycol, or carbohydrates.
“Z” may represent a terminal carboxyl (COOH); an amido group; an ester group (COOR) including, but not limited to, a t-butyloxycarbonyl group; a macromolecular carrier group including, but not limited to, lipid-fatty acid conjugates, polyethylene glycol, or carbohydrates.

TABLE IV
Amino Truncations of HIV-2_{NIHZ} Peptide (SEQ ID NO:5)

X-NWL-Z
X-TNWL-Z
X-FTNWL-Z
X-VFTNWL-Z
X-DVFTNWL-Z
X-WDVFTNWL-Z
X-SWDVFTNWL-Z
X-NSWDVFTNWL-Z
X-LNSWDVFTNWL-Z
X-KLNSWDVFTNWL-Z
X-QKLNSWDVFTNWL-Z
X-LQKLNSWDVFTNWL-Z
X-ELQKLNSWDVFTNWL-Z
X-YELQKLNSWDVFTNWL-Z
X-MYELQKLNSWDVFTNWL-Z
X-NMYELQKLNSWDVFTNWL-Z
X-KNMYELQKLNSWDVFTNWL-Z
X-EKNMYELQKLNSWDVFTNWL-Z
X-QEKNMYELQKLNSWDVFTNWL-Z
X-QQEKNMYELQKLNSWDVFTNWL-Z
X-IQQEKNMYELQKLNSWDVFTNWL-Z
X-QIQQEKNNYELQKLNSWDVFTNWL-Z
X-AQIQQEKNMYELQKLNSWDVFTNWL-Z
X-QAQIQQEKNMYELQKLNSWDVFTNWL-Z
X-EQAQIQQEKNMYELQKLNSWDVFTNWL-Z
X-LEQAQIQQEKIMYELQKLNSWDVFTNWL-Z
X-SLEQAQIQQEKNMYELQKLNSWDVFTNWL-Z
X-QSLEQAQIQQEKNNYELQKLNSWDVFTNWL-Z
X-SQSLEQAQIQQEKNMYELQKLNSWDVFTNWL-Z
X-ISQSLEQAQIQQEKNMYELQKLNSWDVFTNWL-Z
X-NISQSLEQAQIQQEKNMYELQKLNSWDVFTNWL-Z
X-ANISQSLEQAQIQQEKNMYELQKLNSWDVFTNWL-Z
X-EANISQSLEQAQIQQEKNMYELQKLNSWDVFTNWL-Z
X-LEANISQSLEQAQIQQEKNMYELQKLNSWDVFTNWL-Z
The one letter amino acid code is used.
“X” may represent a hydrogen attached to the terminal amino group, an amino protecting group including, but not limited to, carbobenzoxyl, dansyl, or t-butyloxycarbonyl; an acetyl group; a 9-fluorenylmethoxy-carbonyl (FMOC) group; a macromolecular carrier group including, but not limited to, lipid-fatty acid conjugates, polyethylene glycol, or carbohydrates.
“Z” may represent a terminal carboxyl (COOH); an amido group; an ester group (COOR) including, but not limited to, a t-butyloxycarbonyl group; a macromolecular carrier group including, but not limited to, lipid-fatty acid conjugates, polyethylene glycol, or carbohydrates.

Peptides can be synthesized by Genemed Synthesis, Inc., South San Francisco, Calif., using standard solid phase F-Moc chemistry.

### N-Helical Peptides

The amino acid sequence of the N-terminal helix region of HIV_{LAI} is:

ARQLLSGIVQQQNNLLRAIEAQQHLLQLTVWGIKQLQARILAVERYLKDQQLLGI (SEQ ID NO:14)

The peptides of the invention may include peptides comprising SEQ ID NO:14 with or without amino acid insertions which consist of single amino acid residues or stretches of residues ranging from 2 to 15 amino acids in length. One or more insertions may be introduced into the peptide, peptide fragment, analog and/or homolog.

The peptides of the invention may include peptides comprising SEQ ID NO:14 with or without amino acid deletions of the full length peptide, analog, and/or homolog. Such deletions consist of the removal of one or more amino acids from the full-length peptide sequence, with the lower limit length of the resulting peptide sequence being 4 to 6 amino acids. Such deletions may involve a single contiguous portion or greater than one discrete portion of the peptide sequences.

Examples of N-helical Domain Peptide Sequences (all sequences are listed from N-terminus to C-terminus) from different HIV strains include, but are not limited to, the following:

| | |
|---|---|
| HIV-1 Group M: Subtype B Isolate: LAI | |
| ARQLLSGIVQQQNNLLRAIEAQQHLLQLTVWGIKQLQARILAVERYLK | (SEQ ID NO:14) |
| DQQLLGI | |
| SGIVQQQNNLLRAIEAQQHLLQLTVWGIKQLQARILAVERYLKDQ | (SEQ ID NO:50) |
| P-15SGIVQQQNNLLRAIEAQQHLLQLTVWGIKQLQARIL | (SEQ ID NO:51) |
| P-17 NNLLRAIEAQQHLLQLTVWGIKQLQARILAVERYLKDQ | (SEQ ID NO:6) |
| Subtype B Isolate: ADA | |
| SGIVQQQNNLLRAIEAQQHLLQLTVWGIKQLQARVLALERYLRDQ | (SEQ ID NO:52) |
| SGIVQQQNNLLRAIEAQQHLLQLTVWGIKQLQARVL | (SEQ ID NO:53) |
| NNLLRAIEAQQHLLQLTVWGIKQLQARVLALERYLRDQ | (SEQ ID NO:54) |
| SGIVQQQNNLLRAIEAQQRMLQLTVWGIKQLQARVLAVERYLGDQ | (SEQ ID NO:55) |
| SGIVQQQNNLLRAIEAQQRMLQLTVWGIKQLQARVL | (SEQ ID NO:56) |
| NNLLRAIEAQQRMLQLTVWGIKQLQARVLAVERYLGDQ | (SEQ ID NO:57) |
| Subtype B Isolate: 89.6 | |
| SGIVQQQNNLLRAIEAQQHMLQLTVWGIKQLQARVLALERYLRDQ | (SEQ ID NO:58) |
| SGIVQQQNNLLRAIEAQQHMLQLTVWGIKQLQARVL | (SEQ ID NO:59) |
| NNLLRAIEAQQHMLQLTVWGIKQLQARVLALERYLRDQ | (SEQ ID NO:60) |
| Subtype C Isolate: BU910812 | |
| SGIVQQQSNLLRAIEAQQHMLQLTVWGIKQLQARVLAIERYLRDQ | (SEQ ID NO:61) |
| SGIVQQQSNLLRAIEAQQHMLQLTVWGIKQLQARVL | (SEQ ID NO:62) |
| SNLLRAIEAQQHMLQLTVWGIKQLQARVLAIERYLRDQ | (SEQ ID NO:63) |
| Subtype D Isolate: 92UG024D | |
| SGIVQQQNNLLRAIEAQQHLLQLTVWGIKQLQARVLAVESYLKDQ | (SEQ ID NO:64) |
| SGIVQQQNNLLRAIEAQQHLLQLTVWGIKQLQARVL | (SEQ ID NO:65) |
| NNLLRAIEAQQHLLQLTVWGIKQLQARVLAVESYLKDQ | (SEQ ID NO:66) |
| Subtype F Isolate: BZ163A | |
| SGIVQQQSNLLRAIEAQQHLLQLTVWGIKQLQARVLAVERYLQDQ | (SEQ ID NO:67) |
| SGIVQQQSNLLRAIEAQQHLLQLTVWGIKQLQARVL | (SEQ ID NO:68) |
| SNLLRAIEAQQHLLQLTVWGIKQLQARVLAVERYLQDQ | (SEQ ID NO:69) |
| Subtype G Isolate: FI.HH8793 | |
| SGIVQQQSNLLRAIEAQQHLLQLTVWGIKQLQARVLALERYLRDQ | (SEQ ID NO:70) |
| SGIVQQQSNLLRAIEAQQHLLQLTVWGIKQLQARVL | (SEQ ID NO:71) |
| SNLLRAIEAQQHLLQLTVWGIKQLQARVLALERYLRDQ | (SEQ ID NO:72) |
| Subtype H Isolate: BE.VI997 | |
| SGIVQQQSNLLRAIQAQQHMLQLTVWGVKQLQARVLAVERYLKDQ | (SEQ ID NO:73) |
| SGIVQQQSNLLRAIQAQQHMLQLTVWGVKQLQARVL | (SEQ ID NO:74) |
| SNLLRAIQAQQHMLQLTVWGVKQLQARVLAVERYLKDQ | (SEQ ID NO:75) |
| Subtype J Isolate: SE.SE92809 | |
| SGIVQQQSNLLKAIEAQQHLLKLTVWGIKQLQARVLAVERYLKDQ | (SEQ ID NO:76) |
| SGIVQQQSNLLKAIEAQQHLLKLTVWGIKQLQARVL | (SEQ ID NO:77) |
| SNLLKAIEAQQHLLKLTVWGIKQLQARVLAVERYLKDQ | (SEQ ID NO:78) |
| Group N Isolate: CM.YBF30 | |
| SGIVQQQNILLRAIEAQQHLLQLSIWGIKQLQAKVLAIERYLRDQ | (SEQ ID NO:79) |
| SGIVQQQNILLRAIEAQQHLLQLSIWGIKQLQAKVL | (SEQ ID NO:80) |
| NILLRAIEAQQHLLQLSIWGIKQLQAKVLAIERYLRDQ | (SEQ ID NO:81) |
| Group O Isolate: CM.ANT70C | |
| KGIVQQQDNLLRAIQAQQQLLRLSxWGIRQLRARLLALETLLQNQ | (SEQ ID NO:82) |
| KGIVQQQDNLLRAIQAQQQLLRLSxWGIRQLRARL | (SEQ ID NO:83) |
| DNLLRAIQAQQQLLRLSxWGIRQLRARLLALETLLQNQ | (SBQ ID NO:84) |

More specifically, the stabilizing peptides may include peptides corresponding to P-17. P-17 corresponds to residues 558 to 595 of the transmembrane protein gp41 from the HIV-1_{LAI} isolate, and has the 38 amino acid sequence (reading from amino to carboxy terminus):

NH₂-NNLLRAIEAQQHLLQLTVWQIKQLQARILAVERYLKDQ-COOH (SEQ ID NO:6)

In addition to the full-length P-17 38-mer (SEQ ID NO:6), the peptides may include truncations of the P-17 peptide which exhibit stabilizing activity. Such truncated P-17 peptides may comprise peptides of between 3 and 3 8 amino acid residues, i.e., peptides ranging in size from a tripeptide to a 38-mer polypeptide, as shown in Tables V and VI, below. Peptide sequences in these tables are listed from amino (left) to carboxy (right) terminus. “X” may represent an amino group (—NH₂) and “Z” may represent a carboxyl (—COOH) group. Alternatively, “X” and/or “Z” may represent a hydrophobic group, an acetyl group, a FMOC group, an amido group or a covalently attached macromolecular group.

TABLE V
Carboxy Truncations of SEQ ID NO:6

X-NNL-Z
X-NNLL-Z
X-NNLLR-Z
X-NNLLRA-Z
X-NNLLRAI-Z
X-NNLLRAIE-Z
X-NNLLRAIEA-Z
X-NNLLRAIEAQ-Z
X-NNLLRAIEAQQ-Z
X-NNLLRAIEAQQH-Z
X-NNLLRAIEAQQHL-Z
X-NNLLRAIEAQQHLL-Z
X-NNLLRAIEAQQHLLQ-Z
X-NNLLRAIEAQQHLLQL-Z
X-NNLLRAIEAQQHLLQLT-Z
X-NNLLRAIEAQQHLLQLTV-Z
X-NNLLRAIEAQQHLLQLTVW-Z
X-NNLLRAIEAQQHLLQLTVWQ-Z
X-NNLLRAIEAQQHLLQLTVWQI-Z
X-NNLLRAIEAQQHLLQLTVWQIK-Z
X-NNLLRAIEAQQHLLQLTVWQIKQ-Z
X-NNLLRAIEAQQHLLQLTVWQIKQL-Z
X-NNLLRAIEAQQHLLQLTVWQIKQLQ-Z
X-NNLLRAIEAQQHLLQLTVWQIKQLQA-Z
X-NNLLRAIEAQQHLLQLTVWQIKQLQAR-Z
X-NNLLRAIEAQQHLLQLTVWQIKQLQARI-Z
X-NNLLRAIEAQQHLLQLTVWQIKQLQARIL-Z
X-NNLLRAIEAQQHLLQLTVWQIKQLQARTLA-Z
X-NNLLRAIEAQQHLLQLTVWQIKQLQARILAV-Z
X-NNLLRAIEAQQHLLQLTVWQIKQLQARILAVE-Z
X-NNLLRAIEAQQHLLQLTVWQIKQLQARILAVER-Z
X-NNLLRAIEAQQHLLQLTVWQIKQLQARILAVERY-Z
X-NNLLRAIEAQQHLLQLTVWQIKQLQARILAVERYL-Z
X-NNLLRAIEAQQHLLQLTVWQIKQLQARILAVERYLK-Z
X-NNLLRAIEAQQHLLQLTVWQIKQLQARILAVERYLKD-Z
X-NNLLRAIEAQQHLLQLTVWQIKQLQARILAVERYLKDQ-Z+TZ,/32
The one letter amino acid code is used.
“X” may represent a hydrogen attached to the terminal amino group, an amino protecting group including, but not limited to, carbobenzoxyl, dansyl, or t-butyloxycarbonyl; an acetyl group; a 9-fluorenylmethoxy-carbonyl (FMOC) group; a macromolecular carrier group including, but not limited to, lipid-fatty acid conjugates, polyethylene glycol, or carbohydrates.
“Z” may represent a terminal carboxyl (COOH); an amido group; an ester group (COOR) including, but not limited to, a t-butyloxycarbonyl group; a macromolecular carrier group including, but not limited to, lipid-fatty acid conjugates, polyethylene glycol, or carbohydrates.

TABLE VI
Amino Truncations of SEQ ID NO:6

X-KDQ-Z
X-LKDQ-Z
X-YLKDQ-Z
X-RYLKDQ-Z
X-ERYLKDQ-Z
X-VERYLKDQ-Z
X-AVERYLKDQ-Z
X-LAVERYLKDQ-Z
X-ILAVERYLKDQ-Z
X-RILAVERYLKDQ-Z
X-ARILAVERYLKDQ-Z
X-QARILAVERYLKDQ-Z
X-LQARILAVERYLKDQ-Z
X-QLQARILAVERYLKDQ-Z
X-KQLQARILAVERYLKDQ-Z
X-IKQLQARILAVERYLKDQ-Z
X-QIKQLQARILAVERYLKDQ-Z
X-WQIKQLQARILAVERYLKDQ-Z
X-VWQIKQLQARILAVERYLKDQ-Z
X-TVWQIKQLQARILAVERYLKDQ-Z
X-LTVWQIKQLQARILAVERYLKDQ-Z
X-QLTVWQIKQLQARILAVERYLKDQ-Z
X-LQLTVWQTKQLQARILAVERYLKDQ-Z
X-LLQLTVWQIKQLQARILAVERYLKDQ-Z
X-HLLQLTVWQIKQLQARILAVERYLKDQ-Z
X-QHLLQLTVWQIKQLQARILAVERYLKDQ-Z
X-QQHLLQLTVWQIKQLQARILAVERYLKDQ-Z
X-AQQHLLQLTVWQIKQLQARILAVERYLKDQ-Z
X-EAQQHLLQLTVWQIKQLQARILAVERYLKDQ-Z
X-IEAQQHLLQLTVWQIKQLQARTLAVERYLKDQ-Z
X-AIEAQQHLLQLTVWQIKQLQARILAVERYLKDQ-Z
X-RAIEAQQHLLQLTVWQIKQLQARILAVERYLKDQ-Z
X-LRAIEAQQHLLQLTVWQIKQLQARILAVERYLKDQ-Z
X-LLRAIEAQQHLLQLTVWQIKQLQARILAVERYLKDQ-Z
X-NLLRAIEAQQHLLQLTVWQIKQLQARILAVERYLKDQ-Z
X-NNLLRAIEAQQHLLQLTVWQIKQLQARILAVERYLKDQ-Z
The one letter amino acid code is used.
“X” may represent a hydrogen attached to the terminal amino group, an amino protecting group including, but not limited to, carbobenzoxyl, dansyl, or t-butyloxycarbonyl; an acetyl group; a 9-fluorenylmethoxy-carbonyl (FMOC) group; a macromolecular carrier group including, but not limited to, lipid-fatty acid conjugates, polyethylene glycol, or carbohydrates.
“Z”may represent a terminal carboxyl (COOH); an amido group; an ester group (COOR) including, but not limited to, a t-butyloxycarbonyl group; a macromolecular carrier group including, but not limited to, lipid-fatty acid conjugates, polyethylene glycol, or carbohydrates.

The stabilizing peptides also include analogs of P-17 and/or P-17 truncations which may include, but are not limited to, peptides comprising the P-17 sequence (SEQ ID NO:6), or a P-17 truncated sequence, containing one or more amino acid substitutions, insertions and/or deletions. Analogs of P-17 homologs are also within the scope of the invention. The P-17 analogs exhibit disruptive activity, and may possess additional advantageous features, such as, for example, increased bioavailability and/or stability or the ability to stabilize fusion-active structures.

The peptides may further include homologs of P-17 (SEQ ID NO:6) and/or P-17 truncations which exhibit disruptive activity. Such P-17 homologs are peptides whose amino acid sequences are comprised of the amino acid sequences of peptide regions of other, i.e., other than HIV-1_{LAI}, viruses that correspond to the gp41 peptide region from which P-17 (SEQ ID NO:6) was derived. Such viruses may include, but are not limited to, other HIV-1 isolates and HIV-2 isolates.

Amino acid substitutions may be of a conserved or non-conserved nature. Conserved amino acid substitutions consist of replacing one or more amino acids of the P-17 (SEQ ID NO:6) peptide sequence with amino acids of similar charge, size and/or hydrophobicity characteristics, such as, for example, a glutamic acid (E) to aspartic acid (D) amino acid substitution. Non-conserved substitutions consist of replacing one or more amino acids of the P-17 (SEQ ID NO:6) peptide sequence with amino acids possessing dissimilar charge, size and/or hydrophobicity characteristics, such as, for example, a glutamic acid (E) to valine (V) substitution.

Amino acid insertions may consist of single amino acid residues or stretches of residues. The insertions may be made at the carboxy or amino terminal end of the P-17 or P-17 truncated peptide, as well as at a position internal to the peptide. Such insertions will generally range from 2 to 15 amino acids in length. It is contemplated that insertions made at either the carboxy or amino terminus of the peptide of interest may be of a broader size range, with about 2 to about 50 amino acids being preferred. One or more such insertions may be introduced into P-17 (SEQ ID NO:6), P-17 fragments, P-17 analogs and/or P-17 homologs.

Preferred amino or carboxy terminal insertions are peptides ranging from about 2 to about 50 amino acid residues in length, corresponding to gp41 protein regions either amino to or carboxy to the actual P-17 gp41 amino acid sequence, respectively. Thus, a preferred amino terminal or carboxy terminal amino acid insertion would contain gp41 amino acid sequences found immediately amino to or carboxy to the P-17 region of the gp41 protein.

Deletions from P-17 (SEQ ID NO:6), P-17 truncations, P-17 fragments, P-17 analogs and/or P-17 homologs are also within the scope of the invention. Such deletions consist of the removal of one or more amino acids from any of the P-17 peptide sequences, with the lower limit length of the resulting peptide sequence being 4 to 6 amino acids. Such deletions may involve a single contiguous portion of a peptide sequence or greater than one discrete portion of a peptide sequence.

Peptides can be synthesized by Genemed Synthesis, Inc., South San Francisco, Calif., using standard solid phase F-Moc chemistry.

The following examples are not intended to limit the scope of the invention.

### EXAMPLE

### Binding of a Stabilizing Peptide to an Envelope Protein

### Example 1

A version of the P-18 peptide tagged with the influenza hemagglutinin epitope (peptide-YPYDVPDYAGPG (SEQ ID NO:8)) was synthesized and incubated under physiological conditions with envelope-expressing cells with and without soluble CD4 (sCD4). In the presence of sCD4, the tagged peptide (P-18HA) bound to and co-immunoprecipitated gp41 (HXB2 strain) while in the absence of soluble receptor, no complex was observed (FIG. 2A). In similar experiments, co-immunoprecipitation of a recombinant form of gp41 occurred (data not shown).

### Example 2

To confirm the above results, an experiment using a cell expressed (SupTi) form of the CD4 receptor was conducted. As in the previous case, P-18HA complexed gp41 only in the presence of CD4. In addition, the specificity of receptor triggering was confirmed using an anti-CD4 antibody which had been shown to block CD4-gp120 binding. In these experiments, the anti-CD4 antibody blocked complex formation between P-18HA and gp41 (FIG. 2B). In all cases, controls performed as expected. From these results, it can be concluded that P-18 binds to and stabilizes a fusion-active form of gp41.

### Example 3

In a related experiment, it was demonstrated that the C-helical peptide binds envelope protein only after CD4 triggering. This was accomplished using a combination of viral infectivity and cell-cell fusion assays. In the infectivity assay, virus was pretreated with disruptive levels of P-18 which were diluted to sub-disruptive concentrations prior to target cell inoculation. In the cell-cell fusion assay, a similar effect was achieved by pretreating envelope expressing cells with disruptive levels of P-18, followed by washing prior to co-cultivation with CD4+ targets. The effect of the pretreatment was to expose only native (non-fusogenic) envelope (either as cell-free virions or surface expressed envelope) to disruptive levels of peptide. From the result, it could be determined whether the peptide bound to and captured a native or a fusion-active form of envelope protein. In each case, inhibition of virus replication occurred only when P-18 was present at disruptive concentrations at the time of fusion (Furuta, R. A., etal., Nat. Structural Biol. 5:276-279 (1998)). Thus, it can be concluded that the C-helical peptide interacts with a fusion-active form of gp41 which is present only after CD4/gp120 binding.

### Example 4

In an effort to generalize the above observations, a panel of virus isolates were analyzed to determine if different envelopes exhibited different activation requirements. The panel consisted of prototypic and primary virus isolates representing several subtypes and both CXCR4 and CCR5 co-receptor usage. It was discovered that gp41 receptor-mediated activation varied as a function of envelope. It was determined that gp41 activation could be divided into two categories wherein some envelopes required CD4 only and others required both CD4 and chemokine receptor. The prototypic CXCR4, subtype B isolate HXB2 and the primary CCR5, subtype G isolate 92UG975. 10, fell into the first category while the primary CCR5, subtype B isolates SF162 and JR-FL, fell into the second. Representative results from each category are shown in FIG. 2C.

### Example 5—Expression of Recombinant gp41

A fragment of DNA encoding a large portion of the gp41 ectodomain (AA residues 527-670 HXB2 numbering) is generated by PCR amplification from the pSM-WT (HXB2) Env expression plasmid using Taq polymerase and specific primers. This fragment is cloned into a modified form (absent the TrpLE fusion peptide sequence) of the bacterial expression vector pTCLE-G2C, provided by Dr. Terrance Oas, Duke University. The plasmid is based on pAED-4, a T7 expression vector, and was developed specifically for the expression of small proteins (Studier, F. W., et al., Methods Enzymol. 185:60-89 (1990)). The insert is characterized by sequencing and restriction enzyme analysis. The recombinant plasmid containing the gp41 fragment is used to transform BL-21 E. coli host cells. Protein may be expressed and purified using standard procedures (Calderone, T. L., et al., J. Mol. Biol. 262:407-412 (1996)).

### Example 6—Preparation of Fusion-Active rgp41

Fusion-active rgp41 is prepared as follows. The recombinant protein is solubilized in 6M GuHCl at a pH of 7.2 to a concentration of 1.0 mg/ml. The helical peptides (either N or C) are added at an equal molar concentration. The protein-peptide complex is then dialyzed against PBS (using dialysis tubing with a 5000MW cutoff) which will decrease the concentration of denaturant and allow the complex to re-fold. The hybrid complex is then diluted to 200 μg/ml and stored at 4° C. until use.

### Example 7—Preparation of Non-Infectious 8E5/LAV Virus Particles

The 8E5/LAV virus particle is the product of a T-cell clone which contains a single, integrated copy of proviral DNA coding for the synthesis of a defective (non-infectious) HIV-I particle (Folks, T. M., et al., J. Exp. Med. 164:280-290 (1986)). This cell line, 8E5/LAV, was derived from the A3.01 parent cell line (a CD4+ CEM derivative) infected with LAV (now referred to as HIV-lB) by repeated exposure to 5-iodo-2′-deoxyuridine (IUdR). The virus produced by this cloned cell line contained a single base pair addition in the pol gene (position 3241) which gave rise to a non-functional reverse transcriptase resulting in the formation of a non-infectious virus particle (Gendelman, H. E., et al., Virology 160:323-329 (1987)). Thorough characterization of this mutant virus revealed that other structural gene products (gag and env) are produced normally and assemble to form a retroviral particle.

The 8E5/LAV cell line is cultured in RPMI 1640 media supplemented with 10% FCS and antibiotics. A two-day culture of cells at an initial density of 5×10⁵ cells/ml will result in culture supernatant with viral particles at a concentration of about 10⁸/ml (determined by electron microscopy). On the day of harvest, the cells are removed by slow speed centrifugation (1500 RPM) and the culture supernatant is clarified by filtration through a 0.45 μm filter. The viral particles are separated from smaller culture byproducts by ultracentrifugation (26000×g, 5 hours, Sorval TFA 20.250 rotor, 4° C.). The viral pellet is resuspended in a 0.1× volume of PBS and quantified by EM (ABI, Columbia, Md.). The viral particles are stored at −70° C. until use.

### Example 8—Formation of sCD4-Virus-Peptide Complexes

To prepare the immunogen, non-infectious virions are resuspended to a final concentration of about 10⁸ particles/mi in PBS containing the N- or C-peptide at 2 mg/ml. Soluble CD4 (MW 46,000) is added (final concentration 2 mg/ml) and the mixture allowed to incubate at 37° C. for 4 hours. At the end of this time, the mixture of peptide, protein and virus is separated from non-complexed sCD4 and peptide by either size exclusion chromatography (using Sephadex® G-50) or ultracentrifugation on a sucrose gradient.

### Example 9—Purification of Fusion-Active Immunogens from sCD4-Virus-Peptide Complexes

One form of the fusion-active immunogen is recovered following Example 8. A second form is recovered from the dialysis step in Example 6. In generating the second form, the epitope-tagged version of the N- and C-peptides are used to trap the fusion-active complex. Following dialysis, the fusion-active protein/peptide complex is recovered by lysis followed by fractionation (affinity chromatography) using a solid phase modified by the addition of a monoclonal antibody specific for the influenza hemagglutinin epitope. The fusion-active protein/peptide envelope complex is then analyzed by native gel electrophoresis followed by immunoblotting with a combination of gp41 and influenza hemagglutinin antibodies.

### Example 10—Control Experiments and Characterization of Experimental Sera

In addition to immunization with the fusion-active protein/peptide complex, animals are immunized with rgp41 only as a control. The immune response to the peptide-modified regions of gp41 (the N- and C-helices) is determined by a comparison of the control and experimental sera.

Characterization of material derived from immunization with mixtures of sCD4, non-infectious virus and peptide is more complicated. In addition to the CD4/virus/peptide complex, control animals are immunized with sCD4 plus virus and virus alone. In these experiments, antibodies to CD4 and/or the V3 region of the viral envelope confound sample evaluation. Anti-CD4 binding antibodies (which could contribute to virus neutralization) are removed using either affinity chromatography (sCD4-derivatized solid phase) or adsorption of sera with CD4 positive T-cells. Contribution to virus neutralization by anti-V3 antibodies is determined by characterizing experimental samples using both homologous and heterologous virus isolates.

A dramatic difference in neutralizing antibody against divergent isolates indicates a significant contribution by antibodies against the V3 loop. This information plus a side-by-side comparison of experimental and control immunogens allow for an evaluation of the contribution of fusion-active determinants to neutralizing activity.

### Example 11—Immunization with gp41 C-helix peptides

Antibody binding assays can be used to determine the ability of the immunogen vaccines to generate an immune response to various forms of envelope (native vs. denatured). Virus neutralization assays can be used to characterize the antibody response raised against the gp41 domains. The most encouraging results have been from animals immunized with the peptide P-18 modeling the C-helix entry domain (amino acid residues 643-678 of gp41 ). Specifically, two of three animals receiving the immunogen vaccine containing P-18 exhibited a neutralizing antibody response against divergent virus isolates in a variety of assay formats as described below.

Guinea pigs were immunized intramuscularly with 100 μg of P-18 formulated in either Freund's complete (prime) or incomplete (boost) adjuvant. Animals were immunized on days 0, 21, 34, 48 and 62. Blood was collected on days 44,58 and 72. In our initial screen, sera at a 1:10 dilution were tested for the ability to inhibit virus-induced cell killing. In these assays, two of the three animals receiving the P-18 peptide (guinea pigs 233 and 234) were able to block the cytopathic effects of a pair of prototypic HIV-1 isolates. Against the MN isolate, >80% protection was achieved, while against the RF isolate, protection was >50%.

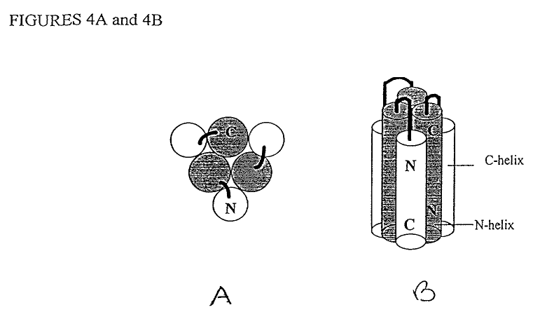

In an assay employing the same format (against HIV-1_{MN}), sera from gp233 and gp234 were titrated. As can be seen in FIG. 4A, these animals displayed the expected dose-related anti-viral activity. Guinea pigs 233 and 234 had a 50% reduction in virus-induced cell killing at 1:40 and 1:37 dilutions, respectively.

In order to confirm these results, a neutralization assay employing a different target cell and endpoint analysis was conducted. In this format, the CEM T-cell line was inoculated with 200 TCID₅₀ of the HIV-1_{MN} isolate. The reduction in viral replication for gp233 and gp234 at a serum dilution of 1:10 is shown in FIG. 4B.

As can be seen, the pattern of virus neutralization observed in the previous assays is repeated here. At this serum dilution, bleed #2 for guinea pigs 233 and 234 gave 80% and 90% virus neutralization, respectively. The same pattern of results was observed against the HIV-1_{SF2} isolate where under identical assay conditions bleed #2 from animals 233 and 234 gave 70% and 50% neutralization, respectively (data not shown). Control animals receiving adjuvant only exhibited no neutralizing activity.

The fact that the sera neutralize the HIV-1 isolates MN, RF and SF2 indicates a breadth of activity unseen in most other subunit immunogens. By comparison, sera generated against V3 peptides are restricted in their activity to a small set of very closely related isolates. Due to the nature of the experiment, the low antibody titers are not unexpected. These animals were immunized with free peptide formulated in Freund's adjuvant. Neither carrier molecules nor accessory proteins were used to enhance the immune response to this molecule. Results from binding assays indicate low, but appreciable levels of antibody against viral envelope. In ELISA assays using recombinant gp41, endpoint titers of 1:6400-1:44,800 were observed for these samples. It is expected that linking P-18 to KLH (or other carrier molecules) and/or administering the envelope protein/peptide complex in an adjuvant designed to enhance the immunogenicity of subunit antigens will result in a significant increase in neutralizing response.

### Example 12—Immune Response

It is of interest to note that the peptide used to generate the novel immune response includes, within its sequence, the linear epitope for the 2F5 monoclonal antibody. To determine if the immune response was against this same region of envelope, or involved a previously unidentified neutralizing epitope, a series of binding experiments were conducted to characterize the reactivity of the polyclonal sera. As can be seen in Table VII, at a dilution of 1:100 all animals exhibit good ELISA binding to the vaccine immunogen (P-18). Sera from these animals also have substantial antibody titers against a peptide derived from the N-terminal P-18 sequence P1 (below). However, when tested at this same dilution against a pair of C-terminal P-18 analogs, P2 and P3 (below), no ELISA reactivity was observed. This result is significant in that the P3 peptide includes the linear binding region ELDKWAS (SEQ ID NO:12) for the 2F5 monoclonal antibody. Based on these results, it can be concluded that the neutralizing activity in the sera is not due to binding to the 2F5 epitome.

TABLE VII
ELISA binding at 1:100 (OD)

| Sample | P1 | P2 | P3 | P-18 |
|---|---|---|---|---|
| gp232-2 | 0.833 | 0.124 | 0.003 | 1.423 |
| gp232-3 | 0.858 | 0.022 | 0.009 | 1.067 |
| gp233-2 | 1.024 | 0.019 | 0.010 | 1.314 |
| gp233-3 | 0.885 | 0.015 | 0.015 | 1.161 |
| gp234-2 | 0.492 | 0.015 | 0.016 | 1.152 |
| gp234-3 | 0.796 | 0.012 | 0.009 | 0.913 |

ELISA binding by guinea pig (gp) sera to P-18 and a set of overlapping peptides corresponding to P-18.

| | |
|---|---|
| P1 YTSLIHSLIEESQNQQEK | (SEQ ID NO:9) |
| P2 EESQNQQEKNEQELLELD | (SEQ ID NO:10) |
| P3 LELDKWASLWNWE | (SEQ ID NO:11) |
| P-18 YTSLIHSLIEESQNQQEKNEQELLELDKWASLWNWF | (SEQ ID NO:1) |

### Example 13—Circular Dichroism Analysis

The effect of point mutations on peptide secondary structure may be carried out as described previously (Wild, C., et al., Proc. Natl. Acad. Sci. USA 89:10537-10541 (1992)). Using circular dichroism (CD), both the type and amount of secondary structure within a peptide or protein can be determined. By CD, a-helical structure is characterized by strong negative signals at 222 and 208 nm. Mean molar elipticity values (determined from peptide concentration and signal strength) provide information on amount (total percent) of a given type of structure. Disruption of secondary structure can be determined by comparing the mean molar elipticity values (derived from the signals at 222 and 208 nm) of the mutant peptide sequences with the wild-type sequence.

### Effect of Mutations on Protein Expression Level

In order to determine the effect of mutations in the context of intact viral envelope on the level of protein expression, the following experiment may be carried out. Briefly, each of the proposed changes are introduced into a wild-type (HXB2) expression vector and the product envelope protein is analyzed for level of expression and loss of gp41 structure.

### Example 14

Preparation of Mutant Envelope Constructs: To generate the desired mutations in the N- and/or C-helical domains, the pSM-WT (HXB2) Env expression plasmid is modified by site-directed mutagenesis from a uridine-substituted single-stranded template (pSM-WT) using the Bio-Rad mutagenesis kit (Bio-Rad Laboratories, Hercules, Calif.). Primers used for mutagenesis are available commercially. Envelope clones containing the desired mutations are identified and confirmed by sequencing using the Sequenase quick denatured plasmid sequencing kit (US Biochemical, Cleveland, Ohio). Following scale-up, the recombinant plasmids are extracted using Qiagen DNA extraction kits and used to transiently transfect 293T cells to study the level of expression and the effect of mutations on gp41 structure.

### Example 15

Level of Envelope Surface Expression: Surface expression of mutant envelope is determined as follows. Envelope expressing cells (293T) are lysed with 0.1 ml of 1% Nonidet P-40 (NP-40), 150 mM NaCl and 100 mM Tris (pH 8.0) buffer (lysis buffer). Approximately 10 μl of the clarified lysate are separated by sodium dodecyl sulfate-polyacrylamide gel electrophoresis (SDS-PAGE) (4 to 12% NuPAGE gels: NOVEX, San Diego, Calif.) and transferred to an ECL nitrocellulose membrane (Amersham, Arlington Heights, Ill.). The membranes are then probed with HIV+human sera at an appropriate dilution in 5% milk-PBS, washed, re-probed with peroxidase-conjugated secondary antibody (Sigma, St. Louis, Mo.) and washed again prior to detection by chemiluminescence (Amersham) and autoradiography.

### Example 16

Surface Immunoprecipitation Assay: Cells expressing mutant envelope are prepared by co-transfection of human 293T cells with a Rev expression vector and the appropriate mutant Env expression vector (prepared as described above in Example 14 by mutagenesis of the pSM-WT (HXB2) Env expression plasmid) using the lipofectamine method (Gibco BRL). Two days following transfection, 5×10⁶ Env-expressing 293T cells are incubated for 1 hour at 37° C. in 0.5 ml Dulbecco's Modified Eagle media (DMEM) in the presence or absence of soluble CD4 (Intracell Inc.) (final concentration 4 μM). Approximately 2 μl of polyclonal sera raised against the six-helix bundle is added and allowed to incubate for an additional hour. Cells are washed twice with phosphate buffered saline (PBS) and lysed with 200 μl of lysis buffer (1% Triton X-100®, 150 mM NaCl, 50 mM Tris-HCl, pH 7.4). The clarified supernatants are incubated 1 hour at 4° C. with a mix of 12.5 μM protein A-Agarose/12.5 μM of protein G-Agarose (GIBCO BRL) followed by washing with lysis buffer (3×). Immunoprecipitated complexes are then analyzed by 10% SDS-PAGE (NOVEX), immunoblotted with anti-gp41 monoclonal antibody Chessie 8 (obtained from NIH AIDS Research and Reference Reagent Program) and detected by chemiluminescence (Amersham) and autoradiography.

### Example 17—Preparation and Bacterial Expression of Mutant gp41 Constructs

Recombinant gp41 containing structure-disrupting mutations are prepared as follows. The pSM-WT (HXB2) Env expression plasmid are modified by site-directed mutagenesis as described above in Example 14 to generate DNA encoding gp41 with N-helix mutations at positions 578 (I to G) or 571 (L to G) & 578 (I to G) or 571 (L to G), 578 (I to G) & 585 (I to G) and C-helix mutations at positions 654 (S to G) or 647 (I to G) & 654 (S to G) or 647 (I to G), 654 (S to G) & 661 (N to G). Mutation-containing fragments corresponding to gp41 amino acid residues 527-670 (HXB2 numbering) are generated by PCR and verified by sequencing. These fragments are subcloned in the expression vector pTCLE-G2C. Protein is expressed and purified using standard procedures (Calderone, T. L., et al., J. Mol. Biol. 262:407-412 (1996)).

Recombinant forms of gp140 (envelope absent the gp120/gp41 cleavage site) containing these same structure-disrupting mutations in the N- or C- helix can also be prepared and purified. This material corresponds to the SF-162 envelope sequence and can be derived from a from stable mammalian (CHO cell lines) expression system.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following claims.

All publications, patents and patent applications mentioned in this specification are indicative of the level of skill of those in the art to which the invention pertains. All publications, patents and patent applications are herein incorporated by reference to the same extent as if each individual publication or patent application was specifically and individually indicated to be incorporated by reference in their entirety.

What is claimed is:

1. An immunogenic composition, comprising:
(a) at least one viral envelope protein or fragment thereof exterior to a viral membrane, and
(b) an amount of at least one stabilizing peptide effective to disrupt formation of one or more structural intermediates necessary for viral fusion and entry, and, optionally,
(c) at least one viral cell surface receptor or fragment thereof, wherein the stabilizing peptide is capable of associating with the envelope protein or fragment thereof to form a stabilized, fusion-active structure.

2. The immunogenic composition of claim 1, wherein the at least one viral envelope protein or fragment thereof is a glycoprotein.

3. The immunogenic composition of claim 2, wherein the glycoprotein is the HIV-1 gp41/gp120 complex.

4. The immunogenic composition of claim 1, wherein the at least one viral cell surface receptor or fragment thereof is an HIV-1 cell surface receptor or a soluble fragment thereof.

5. The immunogenic composition of claim 4, wherein the HIV-1 cell surface receptor or fragment thereof is CD4.

6. The immunogenic composition of claim 1, wherein the at least one stabilizing peptide is selected from the group consisting of: a peptide comprising SEQ ID NO: 1, a peptide comprising a fragment of SEQ ID NO:1, a peptide comprising SEQ ID NO:2, a peptide comprising a fragment of SEQ ID NO:2, a peptide comprising SEQ ID NO:3, a peptide comprising a fragment of SEQ ID NO:3, a peptide comprising SEQ ID NO:4, a peptide comprising a fragment of SEQ ID NO:4, a peptide comprising SEQ ID NO:5, a peptide comprising a fragment of SEQ ID NO:5, a peptide comprising SEQ ID NO:6, a peptide comprising a fragment of SEQ ID NO:6, a peptide comprising SEQ ID NO:7, a peptide comprising a fragment of SEQ ID NO:7, a peptide comprising SEQ ID NO:9, a peptide comprising a fragment of SEQ ID NO:9, a peptide comprising any combination of SEQ ID NOS:1-7 and 9, a peptide comprising any combination of fragments of SEQ ID NOS:1-7 and 9, a peptide functionally equivalent to any one of SEQ ID NOS:1-7 and 9, a homolog of any of SEQ ID NOS:1-7 and 9 and an analog of any of SEQ ID NOS:1-7 and 9.

7. An immunogenic composition, produced by a process comprising:
(a) incubating at least one non-infectious viral particle with a concentration of one or more stabilizing peptides effective to disrupt formation of one or more structural intermediates necessary for viral fusion and entry to obtain a mixture; and
(b) adding a soluble form of one or more viral cell surface receptors or a fragment thereof to the mixture, whereby an immunogenic composition is created.

8. The immunogenic composition of claim 7, comprising at least one viral envelope protein or fragment thereof exterior to the viral membrane, at least one viral cell surface receptor or fragment thereof and an amount of at least one stabilizing peptide effective to disrupt formation of one or more structural intermediates necessary for viral fusion and entry.

9. A method of preparing an immunogenic composition, comprising:
(a) incubating at least one non-infectious viral particle having at least one surface envelope protein or fragment thereof exterior to the viral membrane with an amount of at least one stabilizing peptide effective to disrupt formation of one or more structural intermediates necessary for viral fusion and entry to obtain a protein/peptide first mixture;
(b) adding a soluble form of at least one cell surface receptor or fragment thereof to the protein/peptide first mixture to create a second mixture; and
(c) isolating the resulting fusion-active protein/peptide complex from the second mixture.

10. The method of claim 9, wherein the protein/peptide complex is isolated from the second mixture by treating the second mixture with a detergent.

11. The method of claim 9, further comprising:
(d) purifying the isolated protein/peptide complex.

12. The method of claim 11, wherein the isolated protein/peptide complex is purified by affinity chromatography, ion exchange chromatography, ultracentrifugation or gel filtration.

13. The method of claim 9, wherein the at least one surface envelope protein or fragment thereof is the HIV-1 gp41/gp120 complex.

14. The method of claim 9, wherein the at least one cell surface receptor or fragment thereof is an HIV-1 cell surface receptor.

15. The method of claim 14, wherein the HIV-1 cell surface receptor is CD4.

16. The method of claim 9, wherein the at least one stabilizing peptide is selected from the group consisting of: a peptide comprising SEQ ID NO: 1, a peptide comprising a fragment of SEQ ID NO:1, a peptide comprising SEQ ID NO:2, a peptide comprising a fragment of SEQ ID NO:2, a peptide comprising SEQ ID NO:3, a peptide comprising a fragment of SEQ ID NO:3, a peptide comprising SEQ ID NO:4, a peptide comprising a fragment of SEQ ID NO:4, a peptide comprising SEQ ID NO:5, a peptide comprising a fragment of SEQ ID NO:5, a peptide comprising SEQ ID NO:6, a peptide comprising a fragment of SEQ ID NO:6, a peptide comprising SEQ ID NO:7, a peptide comprising a fragment of SEQ ID NO:7, a peptide comprising SEQ ID NO:9, a peptide comprising a fragment of SEQ ID NO:9, a peptide comprising any combination of SEQ ID NOS:1-7 and 9, a peptide comprising any combination of fragments of SEQ ID NOS:1-7 and 9, a peptide functionally equivalent to any one of SEQ ID NOS:1-7 and 9, a homolog of any of SEQ ID NOS:1-7 and 9 and an analog of any of SEQ ID NOS:1-7 and 9.

17. The method of claim 9, wherein the at least one cell surface receptor is obtained from a cell line that expresses CD4, an appropriate chemokine receptor, or a combination thereof.

18. The method of claim 17, wherein the appropriate chemokine receptor is selected from the group consisting of: CCR5, CXCR4 or a mixture thereof.

19. A method of preparing an immunogenic composition, comprising:
(a) incubating cells expressing at least one HIV envelope protein or fragment thereof exterior to the viral membrane with an amount of at least one stabilizing peptide effective to disrupt formation of one or more structural intermediates necessary for viral fusion and entry to obtain a protein/peptide first mixture;
(b) adding a soluble form of at least one cell surface receptor or fragment thereof to the protein/peptide first mixture to create a second mixture;
(c) isolating the resulting fusion-active protein/peptide complex from the second mixture by treating the second mixture with a lysis buffer; and
(d) purifying the protein/peptide complex.

20. The method of claim 19, wherein the protein/peptide complex is purified by affinity chromatography, ion exchange chromatography, ultracentrifugation or gel filtration.

21. The method of claim 19, wherein the cells expressing the at least one HIV envelope protein or fragment thereof are cells infected with a recombinant vaccinia virus expressing the HIV-1 envelope protein or fragment thereof.

22. The method of claim 19, wherein the at least one stabilizing peptide is selected from the group consisting of: a peptide comprising SEQ ID NO: 1, a peptide comprising a fragment of SEQ ID NO:1, a peptide comprising SEQ ID NO:2, a peptide comprising a fragment of SEQ ID NO:2, a peptide comprising SEQ ID NO:3, a peptide comprising a fragment of SEQ ID NO:3, a peptide comprising SEQ ID NO:4, a peptide comprising a fragment of SEQ ID NO:4, a peptide comprising SEQ ID NO:5, a peptide comprising a fragment of SEQ ID NO:5, a peptide comprising SEQ ID NO:6, a peptide comprising a fragment of SEQ ID NO:6, a peptide comprising SEQ ID NO:7, a peptide comprising a fragment of SEQ ID NO:7, a peptide comprising SEQ ID NO:9, a peptide comprising a fragment of SEQ ID NO:9, a peptide comprising any combination of SEQ ID NOS:1-7 and 9, a peptide comprising any combination of fragments of SEQ ID NOS:1-7 and 9, a peptide functionally equivalent to any one of SEQ ID NOS:1-7 and 9, a homolog of any of SEQ ID NOS:1-7 and 9 and an analog of any of SEQ ID NOS:1-7 and 9.

23. The method of claim 19, wherein the at least one cell surface receptor or fragment thereof is obtained from a cell line that expresses CD4, an appropriate chemokine receptor, or a combination thereof.

24. The method of claim 23, wherein the appropriate chemokine receptor is selected from the group consisting of: CCR5, CXCR4 or a mixture thereof.

25. The method of claim 19, wherein the at least one HIV envelope protein or fragment thereof is a recombinant form of the HIV-1 gp41 ectodomain.

26. The method of claim 19, wherein the protein/peptide complex is formed in the presence of a denaturant.

27. The method of claim 19, wherein the cells expressing the at least one HIV envelope protein or fragment thereof are cells transformed with a vector expressing the HIV-1 envelope protein or fragment thereof.

28. A method of preparing vaccine immunogens comprising isolating gp41 or a fragment thereof and introducing structure disrupting mutations into specific positions in the structural regions of gp41 or fragment thereof resulting in the production of a fusion-active vaccine immunogen.

29. The method of claim 28, wherein the mutations comprise substitutions of the invariant residues within the 4-3 heptad repeats found in each helical region with residues incompatible with the formation of α-helical secondary structure.

30. A product formed by the method of claim 9.
